(19) United States
(12) Patent Application Publication
(10) Pub. No.: US 2024/0294521 A1
(43) Pub. Date: Sep. 5, 2024

(54) CRYSTALLINE FORMS, COMPOSITIONS CONTAINING SAME, AND METHODS OF THEIR USE

(71) Applicant: Repare Therapeutics Inc., St-Laurent (CA)

(72) Inventors: Angelos Dovletoglou, Hamilton, MA (US); Robert Charles Livingston, Carlisle, MA (US); Song Xue, Newton, MA (US); Yuly Chiang Yu, Highland Park, NJ (US); Qi Gao, Franklin Park, NJ (US); Saurin Raval, Piscataway, NJ (US)

(21) Appl. No.: 18/566,492

(22) PCT Filed: Jun. 3, 2022
(86) PCT No.: PCT/CA2022/050892

§ 371 Date: Dec. 1, 2023

Related U.S. Application Data
(60) Provisional application No. 63/197,131, filed on Jun. 4, 2021.

(51) Int. Cl. C07D 471/04; A61K 31/5377

(52) U.S. Cl. CPC C07D 471/04; A61K 31/5377

(57) ABSTRACT

Disclosed are crystalline forms of the compound of the following structure (I): or a sulfate salt, a hemi-sulfate salt, or a hydrogen sulfate salt thereof, or a hydrate thereof. Also disclosed are sulfate salts, hemi-sulfate salts, and hydrogen sulfate salts of the compound of formula (I), and hydrates thereof.

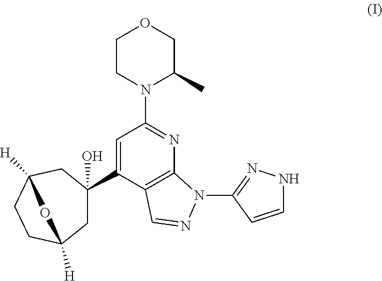 [SMILES: C[C@@H]1COCCN1c1cc([C@]2(O)C[C@H]3CC[C@@H](C2)O3)c2cnn(-c3cc[nH]n3)c2n1]

### FIELD OF THE INVENTION

The invention relates to crystalline forms, pharmaceutical compositions, and their use in the treatment of a disease or condition, e.g., cancer, and, in particular, those diseases or conditions (e.g., cancers) which are dependent on the activity of Ataxia-telangiectasia and RAD-3-related protein (ATR) kinase.

### BACKGROUND

DNA damage occurs continually in cells as a result of environmental insults including ultraviolet radiation, X-rays and endogenous stress factors, such as reactive oxygen and hydrolysis of bases. Cancer cells are subject to a higher rate of DNA damage inherently induced by higher rates of DNA replication in these cells. Several DNA damage response (DDR) pathways have evolved in a highly coordinated manner to help repair DNA damage and to act as a cellular checkpoint to stop the replication of cells with damaged DNA, allowing for repair functions to occur before the damaged DNA is passed on to daughter cells. Each of the identified DNA repair pathways sense and repair distinct but overlapping types of DNA damage.

One major DDR protein that acts as a key cell cycle checkpoint is the ataxia telangiectasia mutated and rad3-related (ATR) kinase, related to the family of phosphoinositide 3-kinase-related protein kinases (PIKKs). ATR is activated by single stranded (ss) DNA lesions caused by stalled replication forks or during nucleotide excision repair but is also activated by double strand breaks following DNA end resection during homologous recombination.

ATR has been identified as an important cancer target since it is essential for dividing cells. Cancer cells that have high levels of replication stress due to oncogenic mutations, dysfunctional G1/S checkpoint control (e.g., loss of p53 function), defects in other DNA repair pathways (e.g., ATM) or that are subject to the effects of DNA damaging agents, e.g., radiation therapy or chemotherapeutic agents, are therefore more dependent on ATR for DNA repair and survival. Together, these results highlight a rationale for the selective sensitivity of proliferating tumor cells to ATR inhibition and the potential for a therapeutic window over healthy proliferating cells.

There is a need for new anti-cancer therapies and, in particular, for ATR inhibitor-based anti-cancer therapies.

### SUMMARY OF THE INVENTION

The invention features crystalline forms, salts, pharmaceutical compositions, and their use in the treatment of a disease or condition (e.g., cancer) which is dependent on the activity of Ataxia-telangiectasia and RAD-3-related protein (ATR) kinase.

In one aspect, the invention provides a crystalline form of a compound of formula (I):

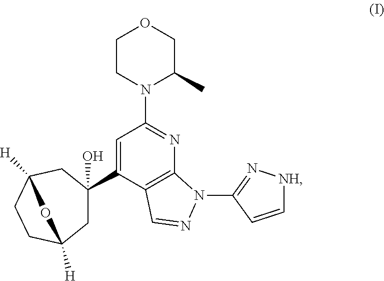 [SMILES: C[C@@H]1COCCN1c1cc([C@]2(O)C[C@H]3CC[C@@H](C2)O3)c2cnn(-c3cc[nH]n3)c2n1]

where the crystalline form is characterized by a powder x-ray diffraction pattern having peaks at 9.6°2θ±0.2°2θ and 15.8°2θ±0.2°2θ. In some embodiments, the crystalline form is further characterized by a powder x-ray diffraction pattern having peaks at 8.9° 2θ±0.2°2θ and 20.5°2θ±0.2°2θ. In some embodiments, the crystalline form is further characterized by a powder x-ray diffraction pattern having peaks at 19.8°2θ±0.2°2θ and 20.7°2θ±0.2°2θ. In some embodiments, the crystalline form is further characterized by a powder x-ray diffraction pattern having peaks at 18.2°2θ±0.2°2θ and 19.2°2θ±0.2°2θ. In some embodiments, the crystalline form is further characterized by a powder x-ray diffraction pattern having peaks at 17.8°2θ±0.2 °2θ, 22.2°2θ±0.2°2θ, and 23.9°2θ±0.2°2θ.

In some embodiments, the crystalline form is further characterized by a differential scanning calorimetry thermogram having an endothermic event onset at 243° C. to 247° C.

In another aspect, the invention provides a crystalline form of a compound of formula (I):

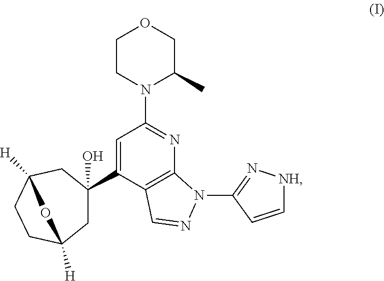 [SMILES: C[C@@H]1COCCN1c1cc([C@]2(O)C[C@H]3CC[C@@H](C2)O3)c2cnn(-c3cc[nH]n3)c2n1]

where the crystalline form is characterized by a powder x-ray diffraction pattern having peaks at 8.1°2θ±0.2°2θ and 20.3°2θ±0.2°2θ. In some embodiments, the crystalline form is further characterized by a powder x-ray diffraction pattern having peaks at 9.1°2θ±0.2°2θ, 15.9°2θ±0.2°2θ, and 23.6°2θ±0.2°2θ. In some embodiments, the crystalline form is further characterized by a powder x-ray diffraction pattern having peaks at 14.3°2θ±0.2°2θ, 16.8°2θ+0.2°2θ, and 19.3°2θ±0.2°2θ. In some embodiments, the crystalline form is further characterized by a powder x-ray diffraction pattern having peaks at 16.1°2θ±0.2°2θ and 21.3°2θ±0.2°2θ. In some embodiments, the crystalline form is further characterized by a powder x-ray diffraction pattern having a peak at 12.3°2θ±0.2°2θ.

In some embodiments, the crystalline form is further characterized by a differential scanning calorimetry thermogram having an endothermic event onset at 82° C. to 109° C. In some embodiments, the crystalline form is further characterized by a differential scanning calorimetry thermogram having an endothermic event onset at 245° C. to 248° C.

In another aspect, the invention provides a crystalline form of a compound of formula (I):

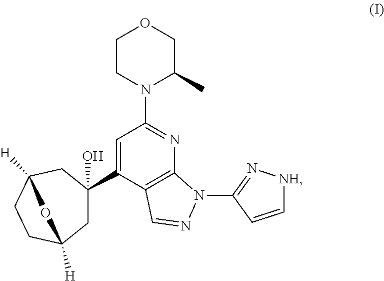 [SMILES: C[C@@H]1COCCN1c1cc([C@]2(O)C[C@H]3CC[C@@H](C2)O3)c2cnn(-c3cc[nH]n3)c2n1]

where the crystalline form is characterized by a powder x-ray diffraction pattern having peaks at 7.2°2θ±0.2°2θ, 20.4°2θ±0.2°2θ, and 29.4°2θ±0.2°2θ. In some embodiments, the crystalline form is further characterized by a powder x-ray diffraction pattern having peaks at 10.2 °2θ±0.2°2θ, 14.4°2θ±0.2°2θ, and 17.2°2θ±0.2°2θ. In some embodiments, the crystalline form is further characterized by a powder x-ray diffraction pattern having peaks at 17.6°20±0.2 °2θ and 27.0°2θ±0.2°2θ. In some embodiments, the crystalline form is further characterized by a powder x-ray diffraction pattern having peaks at 12.5°2θ±0.2°2θ and 30.9°2θ±0.2°2θ. In some embodiments, the crystalline form is further characterized by a powder x-ray diffraction pattern having peaks at 15.4°2θ±0.2°2θ and 18.3°2θ±0.2°2θ. In some embodiments, the crystalline form is further characterized by a powder x-ray diffraction pattern having a peak at 19.7 °2θ±0.2°2θ.

In some embodiments, the crystalline form is further characterized by a differential scanning calorimetry thermogram having an endothermic event onset at 245° C. to 249° C. In some embodiments, the crystalline form is further characterized by a differential scanning calorimetry thermogram having an endothermic event onset at 111° C. to 154° C.

In another aspect, the invention provides a crystalline form of a hydrogen sulfate salt of a compound of formula (I):

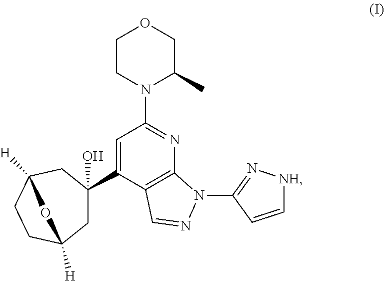 [SMILES: C[C@@H]1COCCN1c1cc([C@]2(O)C[C@H]3CC[C@@H](C2)O3)c2cnn(-c3cc[nH]n3)c2n1]

where the crystalline form is characterized by a powder x-ray diffraction pattern having peaks at 13.0°2θ±0.2°2θ, 19.7°2θ±0.2°2θ, and 25.6°2θ±0.2°2θ. In some embodiments, the crystalline form is further characterized by a powder x-ray diffraction pattern having peaks at 14.8 °2θ±0.2°2θ and 16.5°2θ±0.2°2θ. In some embodiments, the crystalline form is further characterized by a powder x-ray diffraction pattern having peaks at 20.1°2θ±0.2°2θ and 24.5 °2θ±0.2°2θ. In some embodiments, the crystalline form is further characterized by a powder x-ray diffraction pattern having peaks at 14.3°2θ±0.2°2θ, 17.9°2θ±0.2°2θ, and 18.2°2θ±0.2 °2θ. In some embodiments, the crystalline form is further characterized by a powder x-ray diffraction pattern having peaks at 19.3°2θ±0.2°2θ and 21.4°2θ±0.2°2θ. In some embodiments, the crystalline form is further characterized by a powder x-ray diffraction pattern having peaks at 24.6°2θ±0.2°2θ and 25.6°2θ±0.2°2θ.

In some embodiments, the crystalline form is further characterized by a differential scanning calorimetry thermogram having an endothermic event onset at 183° C. to 218° C.

In another aspect, the invention provides a crystalline of form of a hydrate of a hydrogen sulfate salt of a compound of formula (I):

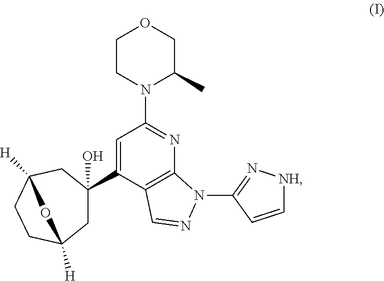 [SMILES: C[C@@H]1COCCN1c1cc([C@]2(O)C[C@H]3CC[C@@H](C2)O3)c2cnn(-c3cc[nH]n3)c2n1]

where the crystalline form is characterized by a powder x-ray diffraction pattern having peaks at 5.9°2θ±0.2°2θ and 11.8°2θ±0.2°2θ. In some embodiments, the crystalline form is further characterized by a powder x-ray diffraction pattern having peaks at 14.9°2θ±0.2°2θ and 20.6 °2θ±0.2°2θ. In some embodiments, the crystalline form is further characterized by a powder x-ray diffraction pattern having peaks at 17.2°2θ±0.2°2θ and 21.2°2θ±0.2°2θ. In some embodiments, the crystalline form is further characterized by a powder x-ray diffraction pattern having peaks at 13.3°2θ±0.2°2θ, 14.2°2θ±0.2°2θ, and 22.0°2θ±0.2°2θ. In some embodiments, the crystalline form is further characterized by a powder x-ray diffraction pattern having a peak at 16.7°2θ±0.2°2θ. In some embodiments, the crystalline form is further characterized by a powder x-ray diffraction pattern having peaks at 24.1°2θ±0.2°2θ and 29.5 °2θ±0.2°2θ.

In some embodiments, the crystalline form is further characterized by a differential scanning calorimetry thermogram having an endothermic event onset at 91° C. to 116° C.

In another aspect, the invention provides a crystalline form of a compound of formula (I):

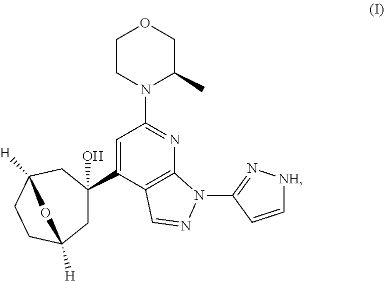 [SMILES: C[C@@H]1COCCN1c1cc([C@]2(O)C[C@H]3CC[C@@H](C2)O3)c2cnn(-c3cc[nH]n3)c2n1]

where the crystalline form is characterized by a powder x-ray diffraction pattern having a peak at 5.5°2θ±0.2°2θ. In some embodiments, the crystalline form is further characterized by a powder x-ray diffraction pattern having a peak at 2.0°2θ±0.2°2θ. In some embodiments, the crystalline form is further characterized by a powder x-ray diffraction pattern having a peak at 8.5°2θ±0.2 °2θ. In some embodiments, the crystalline form is further characterized by a powder x-ray diffraction pattern having a peak at 16.2°2θ±0.2°2θ. In some embodiments, the crystalline form is further characterized by a powder x-ray diffraction pattern having peaks at 19.0°2θ±0.2°2θ and 21.1°2θ±0.2°2θ. In some embodiments, the crystalline form is further characterized by a powder x-ray diffraction pattern having peaks at 12.0°2θ±0.2°2θ and 17.1°2θ±0.2°2θ. In some embodiments, the crystalline form is further characterized by a powder x-ray diffraction pattern having peaks at 9.5°2θ±0.2°2θ and 14.7°2θ±0.2°2θ. In some embodiments, the crystalline form is further characterized by a powder x-ray diffraction pattern having a peak at 18.7 °2θ±0.2°2θ. In some embodiments, the crystalline form is further characterized by a powder x-ray diffraction pattern having a peak at 18.4°2θ±0.2°2θ.

In some embodiments, the crystalline form is further characterized by a differential scanning calorimetry thermogram having an endothermic event onset at 252° C. to 253° C.

In another aspect, the invention provides a crystalline of form of a hemi-sulfate salt of a compound of formula (I):

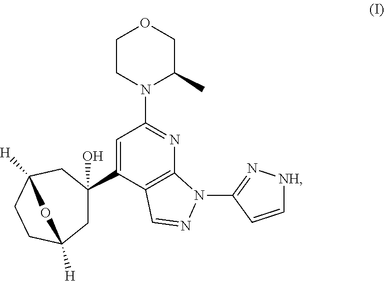 [SMILES: C[C@@H]1COCCN1c1cc([C@]2(O)C[C@H]3CC[C@@H](C2)O3)c2cnn(-c3cc[nH]n3)c2n1]

wherein the crystalline form is characterized by a powder x-ray diffraction pattern having peaks at 8.3°2θ±0.2°2θ and 15.6°2θ±0.2°2θ. In some embodiments, the crystalline form is further characterized by a powder x-ray diffraction pattern having peaks at 15.1°2θ±0.2°2θ and 23.4 °2θ±0.2°2θ. In some embodiments, the crystalline form is further characterized by a powder x-ray diffraction pattern having a peak at 17.4°2θ±0.2°2θ. In some embodiments, the crystalline form is further characterized by a powder x-ray diffraction pattern having a peak at 17.9°2θ±0.2 °2θ.

In some embodiments, the crystalline form is further characterized by a differential scanning calorimetry thermogram having an endothermic event onset at 171° C. to 183° C.

In a further aspect, the invention provides a hydrogen sulfate salt of a compound of formula (I):

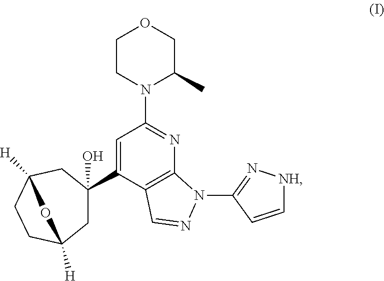 [SMILES: C[C@@H]1COCCN1c1cc([C@]2(O)C[C@H]3CC[C@@H](C2)O3)c2cnn(-c3cc[nH]n3)c2n1]

or a hydrate thereof.

In yet further aspect, the invention provides a hemi-sulfate salt of a compound of formula (I):

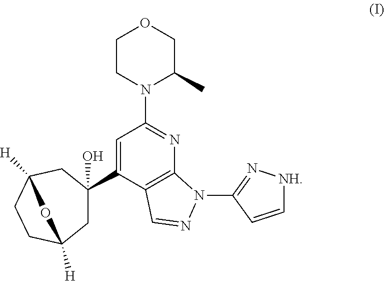 [SMILES: C[C@@H]1COCCN1c1cc([C@]2(O)C[C@H]3CC[C@@H](C2)O3)c2cnn(-c3cc[nH]n3)c2n1]

In still further aspect, the invention provides a sulfate salt of a compound of formula (I):

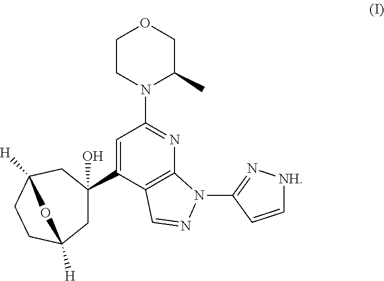 [SMILES: C[C@@H]1COCCN1c1cc([C@]2(O)C[C@H]3CC[C@@H](C2)O3)c2cnn(-c3cc[nH]n3)c2n1]

In yet another aspect, the invention provides a pharmaceutical composition including any one of the crystalline forms described herein or any one of the salts described herein.

In a further aspect, the invention provides a method of inhibiting ATR kinase in a cell expressing ATR kinase, the method including contacting the cell with an effective amount of any one of the crystalline forms described herein, the pharmaceutical composition described herein, or any one of the salts described herein. In some embodiments, the cell is in a subject.

In yet a further aspect, the invention provides a method of treating a subject in need thereof comprising administering to the subject an effective amount of any one of the crystalline forms described herein, the pharmaceutical composition described herein, or any one of the salts described herein. In some embodiments, the subject is suffering from, and is in need of a treatment for, a disease or condition having the symptom of cell hyperproliferation (e.g., a cancer (e.g., a carcinoma (e.g., medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, Schneiderian carcinoma, scirrhous carcinoma, carcinoma scroti, signet ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum), sarcoma (e.g., chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telang iectaltic sarcoma), adenocarcinoma, leukemia (e.g., nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, undifferentiated cell leukemia), or melanoma (e.g., acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, superficial spreading melanoma))).

In some embodiments, the cancer is prostate cancer, thyroid cancer, endocrine system cancer, brain cancer, breast cancer, cervix cancer, colon cancer, head & neck cancer, liver cancer, kidney cancer, lung cancer, non-small cell lung cancer, melanoma, mesothelioma, ovarian cancer, sarcoma, stomach cancer, uterus cancer, medulloblastoma, ampullary cancer, colorectal cancer, or pancreatic cancer.

In some embodiments, the cancer is Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphoma, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

In some embodiments, the subject is suffering from, and is in need of a treatment for, a pre-malignant condition.

### Definitions

The term “adenocarcinoma,” as used herein, represents a malignancy of the arising from the glandular cells that line organs within an organism. Non-limiting examples of adenocarcinomas include non-small cell lung cancer, prostate cancer, pancreatic cancer, esophageal cancer, and colorectal cancer.

The term “ATR kinase,” as used herein, refers to Ataxia-telangiectasia and RAD-3-related protein kinase.

The term “cancer,” as used herein, refers to all types of cancer, neoplasm or malignant tumors found in mammals (e.g., humans), including leukemia, carcinomas and sarcomas. Non limiting examples of cancers that may be treated with a compound or method provided herein include prostate cancer, thyroid cancer, endocrine system cancer, brain cancer, breast cancer, cervix cancer, colon cancer, head & neck cancer, liver cancer, kidney cancer, lung cancer, non-small cell lung cancer, melanoma, mesothelioma, ovarian cancer, sarcoma, stomach cancer, uterus cancer, medulloblastoma, ampullary cancer, colorectal cancer, and pancreatic cancer.

Additional non-limiting examples may include, Hodgkin's disease, Non-Hodgkin's lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphoma, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, and prostate cancer.

The term “carcinoma,” as used herein, refers to a malignant new growth made up of epithelial cells tending to infiltrate the surrounding tissues and give rise to metastases. Non limiting examples of carcinomas that may be treated with a compound or method provided herein include, e.g., medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, Schneiderian carcinoma, scirrhous carcinoma, carcinoma scroti, signet ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “crystalline form B,” as used herein, refers to a crystalline form of a compound of formula (1):

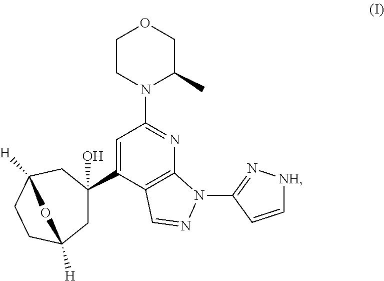 [SMILES: C[C@@H]1COCCN1c1cc([C@]2(O)C[C@H]3CC[C@@H](C2)O3)c2cnn(-c3cc[nH]n3)c2n1]

where the crystalline form is characterized by a powder x-ray diffraction pattern having peaks at 9.6°2θ±0.2°2θ and 15.8°2θ±0.2°2θ. In some embodiments, crystalline form B is further characterized by a powder x-ray diffraction pattern having peaks at 8.9°2θ±0.2°2θ and 20.5°2θ±0.2°2θ. In some embodiments, crystalline form B is further characterized by a powder x-ray diffraction pattern having peaks at 19.8°2θ±0.2°2θ and 20.7°2θ±0.2°2θ. In some embodiments, crystalline form B is further characterized by a powder x-ray diffraction pattern having peaks at 18.2°2θ±0.2°2θ and 19.2°2θ±0.2°2θ. In some embodiments, crystalline form B is further characterized by a powder x-ray diffraction pattern having peaks at 17.8°2θ±0.2°2θ, 22.2°2θ±0.2°2θ, and 23.9°2θ±0.2°2θ. In some embodiments, crystalline form B is further characterized by a differential scanning calorimetry thermogram having an endothermic event onset at 243° C. to 247° C.

The term “crystalline form C,” as used herein, refers to a crystalline form of a compound of formula (I):

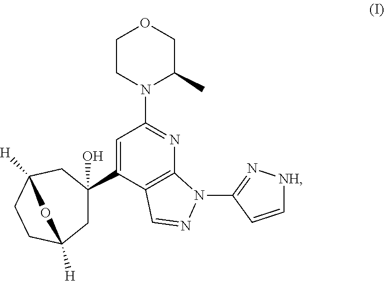 [SMILES: C[C@@H]1COCCN1c1cc([C@]2(O)C[C@H]3CC[C@@H](C2)O3)c2cnn(-c3cc[nH]n3)c2n1]

where the crystalline form is characterized by a powder x-ray diffraction pattern having peaks at 8.1°20±0.2°20 and 20.3°20±0.2 026. In some embodiments, crystalline form C is characterized by a powder x-ray diffraction pattern having peaks at 9.1°2θ±0.2°2θ, 15.9°2θ±0.2°2θ, and 23.6°2θ±0.2°2θ. In some embodiments, crystalline form C is characterized by a powder x-ray diffraction pattern having peaks at 14.3°2θ±0.2°2θ, 16.8°2θ±0.2°2θ, and 19.3 °2θ±0.2°2θ. In some embodiments, crystalline form C is characterized by a powder x-ray diffraction pattern having peaks at 16.1°2θ±0.2°2θ and 21.3°2θ±0.2°2θ. In some embodiments, crystalline form C is characterized by a powder x-ray diffraction pattern having a peak at 12.3°2θ±0.2°2θ. In some embodiments, crystalline form C is characterized by a differential scanning calorimetry thermogram having an endothermic event onset at 82° C. to 109° C. In some embodiments, crystalline form C is characterized by a differential scanning calorimetry thermogram having an endothermic event onset at 245° C. to 248° C.

The term “crystalline form F,” as used herein, refers to a crystalline form of a compound of formula (I):

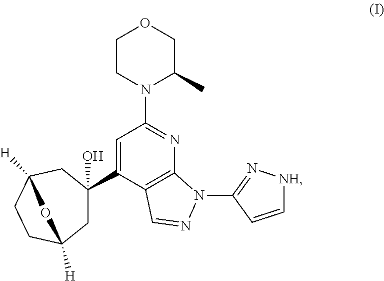 [SMILES: C[C@@H]1COCCN1c1cc([C@]2(O)C[C@H]3CC[C@@H](C2)O3)c2cnn(-c3cc[nH]n3)c2n1]

where the crystalline form is characterized by a powder x-ray diffraction pattern having peaks at 7.2°2θ±0.2°2θ, 20.4°2θ±0.2°2θ, and 29.4°2θ±0.2°2θ. In some embodiments, crystalline form F is further characterized by a powder x-ray diffraction pattern having peaks at 10.2°2θ±0.2 °2θ, 14.4°2θ±0.2°2θ, and 17.2°2θ±0.2°2θ. In some embodiments, crystalline form F is further characterized by a powder x-ray diffraction pattern having peaks at 17.6°20±0.2°20 and 27.0 °2θ±0.2°2θ. In some embodiments, crystalline form F is further characterized by a powder x-ray diffraction pattern having peaks at 12.5°2θ±0.2°2θ and 30.9°2θ±0.2°2θ. In some embodiments, crystalline form F is further characterized by a powder x-ray diffraction pattern having peaks at 15.4°2θ±0.2°2θ and 18.3°2θ±0.2°2θ. In some embodiments, crystalline form F is further characterized by a powder x-ray diffraction pattern having a peak at 19.7°2θ±0.2°2θ.

In some embodiments, crystalline form F is further characterized by a differential scanning calorimetry thermogram having an endothermic event onset at 245° C. to 249° C. In some embodiments, crystalline form F is further characterized by a differential scanning calorimetry thermogram having an endothermic event onset at 111° C. to 154° C.

The term “crystalline form M,” as used herein, refers to a crystalline form of a compound of formula (I):

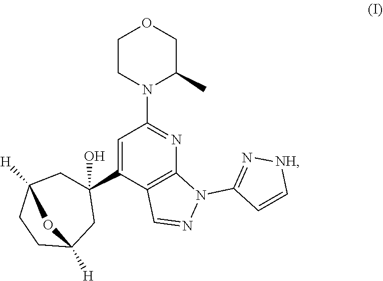

where the crystalline form is characterized by a powder x-ray diffraction pattern having a peak at 5.5°2θ±0.2°2θ. In some embodiments, the crystalline form is further characterized by a powder x-ray diffraction pattern having a peak at 2.0°2θ±0.2°2θ. In some embodiments, crystalline form M is further characterized by a powder x-ray diffraction pattern having a peak at 8.5°2θ±0.2°2θ. In some embodiments, crystalline form M is further characterized by a powder x-ray diffraction pattern having a peak at 16.2°2θ±0.2°2θ. In some embodiments, crystalline form M is further characterized by a powder x-ray diffraction pattern having peaks at 19.0°2θ±0.2°2θ and 21.1 °2θ±0.2°2θ. In some embodiments, crystalline form M is further characterized by a powder x-ray diffraction pattern having peaks at 12.0°2θ±0.2°2θ and 17.1°2θ±0.2°2θ. In some embodiments, crystalline form M is further characterized by a powder x-ray diffraction pattern having peaks at 9.5°2θ±0.2°2θ and 14.7°2θ±0.2°2θ. In some embodiments, crystalline form M is further characterized by a powder x-ray diffraction pattern having a peak at 18.7°2θ±0.2 °2θ. In some embodiments, crystalline form M is further characterized by a powder x-ray diffraction pattern having a peak at 18.4°2θ±0.2°2θ. In some embodiments, crystalline form M is further characterized by a differential scanning calorimetry thermogram having an endothermic event onset at 252° C. to 253° C.

The term “crystalline hydrogen sulfate form A,” as used herein, refers to a crystalline form of a hydrogen sulfate salt of a compound of formula (I):

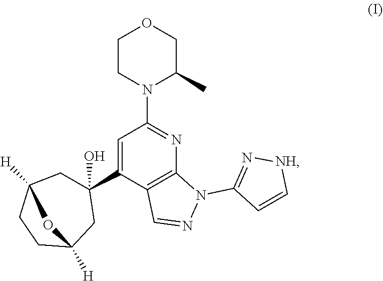

where the crystalline form is characterized by a powder x-ray diffraction pattern having peaks at 13.0°2θ±0.2°2θ, 19.7°2θ±0.2°2θ, and 25.6°2θ±0.2°2θ. In some embodiments, crystalline hydrogen sulfate form A is further characterized by a powder x-ray diffraction pattern having peaks at 14.8°2θ±0.2°2θ and 16.5°2θ±0.2°2θ. In some embodiments, crystalline hydrogen sulfate form A is further characterized by a powder x-ray diffraction pattern having peaks at 20.1°2θ±0.2 °2θ and 24.5°2θ±0.2°2θ. In some embodiments, crystalline hydrogen sulfate form A is further characterized by a powder x-ray diffraction pattern having peaks at 14.3°2θ±0.2°2θ, 17.9°2θ±0.2°2θ, and 18.2°2θ±0.2°2θ. In some embodiments, crystalline hydrogen sulfate form A is further characterized by a powder x-ray diffraction pattern having peaks at 19.3°2θ±0.2°2θ and 21.4°2θ±0.2°2θ. In some embodiments, crystalline hydrogen sulfate form A is further characterized by a powder x-ray diffraction pattern having peaks at 24.6°2θ±0.2 026 and 25.6 °2θ±0.2°2θ. In some embodiments, crystalline hydrogen sulfate form A is further characterized by a differential scanning calorimetry thermogram having an endothermic event onset at 183° C. to 218° C.

The term “crystalline hydrogen sulfate form B,” as used herein, refers to a crystalline form of a hydrate of a hydrogen sulfate salt of a compound of formula (I):

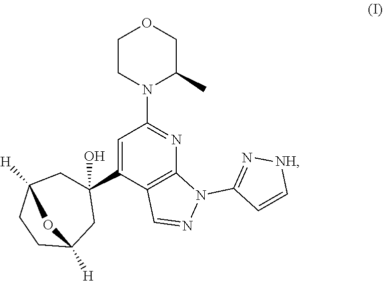

where the crystalline form is characterized by a powder x-ray diffraction pattern having peaks at 5.9°2θ±0.2°20 and 11.8°2θ±0.2°2θ. In some embodiments, crystalline hydrogen sulfate form B is further characterized by a powder x-ray diffraction pattern having peaks at 14.9°2θ±0.2°2θ and 20.6°2θ±0.2°2θ. In some embodiments, crystalline hydrogen sulfate form B is further characterized by a powder x-ray diffraction pattern having peaks at 17.2°2θ±0.2°2θ and 21.2 °2θ±0.2°2θ. In some embodiments, crystalline hydrogen sulfate form B is further characterized by a powder x-ray diffraction pattern having peaks at 13.3°2θ±0.2°2θ, 14.2°2θ±0.2°2θ, and 22.0°2θ±0.2°2θ. In some embodiments, crystalline hydrogen sulfate form B is further characterized by a powder x-ray diffraction pattern having a peak at 16.7°2θ±0.2°2θ. In some embodiments, crystalline hydrogen sulfate form B is further characterized by a powder x-ray diffraction pattern having peaks at 24.1°2θ±0.2°2θ and 29.5°2θ±0.2°2θ. In some embodiments, crystalline hydrogen sulfate form B is further characterized by a differential scanning calorimetry thermogram having an endothermic event onset at 91° C. to 116° C.

The term “crystalline sulfate form A,” as used herein, refers to a crystalline form of a hemi-sulfate salt of a compound of formula (I):

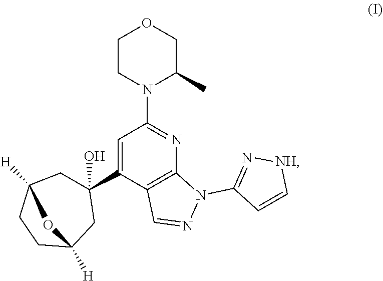

where the crystalline form is characterized by a powder x-ray diffraction pattern having peaks at 8.3°2θ±0.2°20 and 15.6°2θ±0.2°2θ. In some embodiments, the crystalline form is further characterized by a powder x-ray diffraction pattern having peaks at 15.1°2θ±0.2°2θ and 23.4 °2θ±0.2°2θ. In some embodiments, the crystalline form is further characterized by a powder x-ray diffraction pattern having a peak at 17.4°2θ±0.2°2θ. In some embodiments, the crystalline form is further characterized by a powder x-ray diffraction pattern having a peak at 17.9°2θ±0.2 °2θ. In some embodiments, the crystalline form is further characterized by a differential scanning calorimetry thermogram having an endothermic event onset at 171° C. to 183° C.

“Disease” or “condition” refer to a state of being or health status of a patient or subject capable of being treated with the compounds or methods provided herein.

The term “leukemia,” as used herein, refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, e.g., acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “lymphoma,” as used herein, refers to a cancer arising from cells of immune origin. Non-limiting examples of T and B cell lymphomas include non-Hodgkin lymphoma and Hodgkin disease, diffuse large B-cell lymphoma, follicular lymphoma, mucosa-associated lymphatic tissue (MALT) lymphoma, small cell lymphocytic lymphoma-chronic lymphocytic leukemia, Mantle cell lymphoma, mediastinal (thymic) large B-cell lymphoma, lymphoplasmacytic lymphoma-Waldenstrom macroglobulinemia, peripheral T-cell lymphoma (PTCL), angioimmunoblastic T-cell lymphoma (AITL)/follicular T-cell lymphoma (FTCL), anaplastic large cell lymphoma (ALCL), enteropathy-associated T-cell lymphoma (EATL), adult T-cell leukemia/lymphoma (ATLL), or extranodal NK/T-cell lymphoma, nasal type.

The term “melanoma,” as used herein, is taken to mean a tumor arising from the melanocytic system of the skin and other organs. Melanomas that may be treated with a compound or method provided herein include, e.g., acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable excipient” or “pharmaceutically acceptable carrier,” as used interchangeably herein, refers to any ingredient other than the compounds described herein (e.g., a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pre-malignant” or “pre-cancerous,” as used herein, refers to a condition that is not malignant but is poised to become malignant. Non-limiting examples of pre-malignant conditions include myelodysplastic syndrome, polyps in the colon, actinic keratosis of the skin, dysplasia of the cervix, metaplasia of the lung, and leukoplakia.

The term “sarcoma” generally refers to a tumor which is made up of a substance like the embryonic connective tissue and is generally composed of closely packed cells embedded in a fibrillar or homogeneous substance. Non-limiting examples of sarcomas that may be treated with a compound or method provided herein include, e.g., a chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer.

“Treatment” and “treating,” as used herein, refer to the medical management of a subject with the intent to improve, ameliorate, stabilize, prevent, or cure a disease or condition. This term includes active treatment (treatment directed to improve the disease or condition); causal treatment (treatment directed to the cause of the associated disease or condition); palliative treatment (treatment designed for the relief of symptoms of the disease or condition); preventative treatment (treatment directed to minimizing or partially or completely inhibiting the development of the associated disease or condition); and supportive treatment (treatment employed to supplement another therapy).

### BRIEF DESCRIPTION OF THE DRAWINGS

FIG. 1 is a microscopic image of the crystals of crystalline form B of the compound of formula (1) isolated from ethyl acetate. The image was made in polarized, visible light.

FIG. 2 is an XRPD diffractogram of crystalline form B of the compound of formula (I) isolated from ethyl acetate.

FIG. 3 is a plot showing TG and DSC traces for crystalline form B of the compound of formula (1) isolated from ethyl acetate.

FIG. 4 is a DVS plot of crystalline form B of the compound of formula (I).

FIGS. 5A and 5B are a series of microscopic images of crystalline form C of the compound of formula (I). FIG. 5A is an image of solids prepared from acetone/water after being vacuum dried at room temperature (crystalline form C). FIG. 5B is an image of crystals (crystalline form C) isolated from ethanol/water. The images were made in polarized, visible light.

FIG. 6 is an XRPD diffractogram of the crystalline form C of the compound of formula (I) isolated from acetone/water.

FIG. 7 is a plot showing TG and DSC traces for crystalline form C of the compound of formula (I) (crystalline form C) isolated from acetone/water.

FIG. 8 is a DVS plot of crystalline form C of the compound of formula (I) isolated from acetone-water.

FIG. 9 is an overlay of XRPD diffractograms comparing the XRPD traces of crystalline form C of the compound of formula (I) (top diffractogram), crystalline form C of the compound of formula (I) after DVS (middle diffractogram), and crystalline form J of the compound of formula (I) (bottom diffractogram).

FIG. 10 is a microscopic image of crystalline form F of the compound of formula (I) previously isolated from aqueous ethanol at room temperature as crystalline form C which later converted to cube-like crystals of crystalline form F. The image was made in polarized, visible light.

FIG. 11 is an XRPD diffractogram of the crystalline form F of the compound of formula (I) previously isolated from aqueous ethanol at room temperature as crystalline form C which later converted to crystalline form F.

FIG. 12 is a plot showing TG and DSC traces for crystalline form F of the compound of formula (1).

FIG. 13 is a DVS plot of crystalline form F of the compound of formula (I).

FIG. 14 is a plot showing an open pan DSC of crystalline form F of the compound of formula (1). Dehydration was observed (1^{st} endotherm with onset at 105° C.), with melting of the anhydrous phase (2^{nd} endotherm with onset at 180° C.), followed by crystallization of a neat form (exotherm with onset at 204° C.) which melted at 245° C. to 248° C.

FIG. 15 is an overlay of XRPD diffractograms comparing the XRPD traces of the simulated pattern of crystalline form F of the compound of formula (I) (from SCXRD, top diffractogram), the XRPD trace of crystalline form F after being heated to 170° C. (second diffractogram from the top), the XRPD trace of crystalline form F after being heated to 190° C. (third diffractogram from the top), the XRPD trace of crystalline form F after being heated to 195° C. (fourth diffractogram from the top), the XRPD trace of crystalline form F after being heated to 235° C. (fifth diffractogram from the top), and a reference XRPD trace of crystalline form B.

FIG. 16 is a microscopic image of crystalline form F after being heated to 195° C., showing some particles containing cracks or lacking birefringence. The image was made in polarized, visible light.

FIG. 17 is plot showing modulated DSC traces for crystalline form F of the compound of formula (I) after being heated to 195° C. A glass transition at 157° C. (midpoint) was observed.

FIG. 18 is an overlay of XRPD diffractograms comparing the XRPD traces of crystalline form F of the compound of formula (I) in water (top diffractogram), the simulated pattern (from SCXRD) of crystalline form F for reference (second diffractogram from the top), the XRPD trace of crystalline form F after the sample was suspended in water for one day (third diffractogram from the top), and the simulated pattern (from SCXRD) of the crystalline form C form for reference.

FIGS. 19A and 19B are a series of microscopic images of two crystalline forms (crystalline forms B and F, respectively) of the compound of formula (I) after being suspended in water for five days at room temperature. FIG. 19A shows that crystalline form B converted to crystalline form C.

FIG. 19B shows that crystalline form F was unchanged. The images were made in polarized, visible light.

FIG. 20 is a microscopic image of the crystalline hydrogen sulfate form of the compound of formula (1) (crystalline hydrogen sulfate form A) isolated from ethanol/tetrahydrofuran. The image was made in polarized, visible light.

FIG. 21 is an XRPD diffractogram of the crystalline hydrogen sulfate form of the compound of formula (I) (crystalline hydrogen sulfate form A) isolated from ethanol/tetrahydrofuran after being oven vacuum dried at room temperature.

FIG. 22 is a plot showing TG and DSC traces for the crystalline hydrogen sulfate form of the compound of formula (I) (crystalline hydrogen sulfate form A).

FIG. 23 is a DVS plot of the crystalline hydrogen sulfate form of the compound of formula (I) (crystalline hydrogen sulfate form A).

FIG. 24 is an overlay of XRPD diffractograms comparing the XRPD traces of the crystalline hydrogen sulfate form of the compound of formula (I) (crystalline hydrogen sulfate form A) after DVS (top diffractogram), the crystalline hydrogen sulfate form A before DVS, and the simulated pattern of the anhydrous hydrogen sulfate salt of the compound of formula (I) from SCXRD.

FIGS. 25A and 25B are a series of microscopic images of a crystalline hydrate of the hydrogen sulfate salt of the compound of formula (I) (crystalline hydrogen sulfate form B). The images were made in polarized, visible light.

FIG. 26 is an XRPD diffractogram of the crystalline hydrate of the hydrogen sulfate salt of the compound of formula (I) (crystalline hydrogen sulfate form B).

FIG. 27 is a plot showing TG and DSC traces for the crystalline hydrate of the hydrogen sulfate salt of the compound of formula (I) (crystalline hydrogen sulfate form B).

FIG. 28 is a DVS plot of the crystalline hydrate of the hydrogen sulfate salt of the compound of formula (I) (crystalline hydrogen sulfate form B).

FIGS. 29A, 29B, 30A, and 30B are a series of microscopic images used to monitor a crystalline metastable form of the hydrogen sulfate salt of the compound of formula (I). The crystalline particles (FIGS. 29A and 29B) had a fine needle-like morphology, and a different XRPD pattern compared to crystalline hydrogen sulfate form A. While stirring in ethanol, after 4 hours a proportion of the particles converted to square-like crystals (FIG. 30A). After stirring for 18 hours in ethanol, the metastable form had fully converted (FIG. 30B) to crystalline hydrogen sulfate form A as confirmed by XRPD analysis.

FIG. 31 is an overlay of XRPD diffractograms comparing the XRPD traces of the crystalline metastable form of the hydrogen sulfate salt of the compound of formula (I) (top diffractogram), crystalline hydrogen sulfate form A (middle diffractogram), and the simulated pattern (from SCXRD) of crystalline hydrogen sulfate form A (bottom diffractogram) for reference.

FIG. 32 is an overlay of XRPD diffractograms comparing the XRPD traces of the crystalline dichloromethane solvate of the compound of formula (I) (obtained after suspending the compound of formula (I) in dichloromethane for three days) after oven drying (48° C., overnight) (top diffractogram), the crystalline dichloromethane solvate of the compound of formula (I) after stirring in dichloromethane (middle diffractogram), and the simulated pattern (from SCXRD) of the crystalline dichloromethane solvate of the compound of formula (I) (bottom diffractogram).

FIG. 33 is an XRPD diffractogram of the crystalline tetrahydrofuran solvate of the compound of formula (I).

FIG. 34 is an XRPD diffractogram of the crystalline methanol solvate of the compound of formula (1).

FIG. 35 is an overlay of XRPD diffractograms comparing the XRPD traces of the crystalline 2-methyl tetrahydrofuran solvate of the compound of formula (I) (obtained after suspending the compound of formula (I) in 2-methyl tetrahydrofuran) after oven drying (48° C., overnight) (top diffractogram), the crystalline 2-methyl tetrahydrofuran (2-MeTHF) solvate of the compound of formula (I) for reference (second diffractogram from the top), the crystalline 2-MeTHF solvate after stirring in 2-MeTHF at 50° C., overnight, then for an additional 2 days at room temperature (third diffractogram from the top), and the crystalline 2-MeTHF solvate after being stirred overnight in 2-MeTHF at 50° C. (bottom diffractogram).

FIG. 36 is an overlay of the XRPD diffractograms for 7-day stability assessment of crystalline form F of the compound of formula (I) in 0.5% methylcellulose (v/v) (400 cps)/0.02% sodium lauryl sulfate (v/v) at room temperature. No form change was obtained after 7 days by XRPD.

FIG. 37 is an overlay of the XRPD diffractograms for stability assessment of the crystalline hydrogen sulfate salt of the compound of formula (I) (crystalline hydrogen sulfate form A). After 4 hours, crystalline form C was observed.

FIG. 38 is an overlay of the XRPD diffractograms for stability assessment of the crystalline hydrate of the hydrogen sulfate salt of the compound of formula (I) (crystalline hydrogen sulfate form B). After stirring overnight in 0.5% methylcellulose (v/v) (400 cps)/0.02% sodium lauryl sulfate (v/v) in water (with and without citrate buffer), crystalline hydrogen sulfate form B was observed to convert to crystalline form C of the compound of formula (I).

FIG. 39 is an overlay of the XRPD diffractograms for 7-day stability assessment of crystalline hydrogen sulfate form A.

FIG. 40 is an overlay of the XRPD diffractograms for 7-day stability assessment of crystalline form F.

FIG. 41 is an overlay of the XRPD diffractograms for 14-day stability assessment of crystalline form B.

FIG. 42 is an overlay of the XRPD diffractograms for 14-day stability assessment of crystalline form F.

FIG. 43 is an overlay of the XRPD diffractograms for 4-week stability assessment of crystalline hydrogen sulfate form A.

FIG. 44 is an overlay of DSC traces of crystalline hydrogen sulfate form A before and after exposure to 25° C./60% relative humidity (RH) and 40° C./75% RH (open and closed vial) after seven days.

FIG. 45 is an overlay of DSC traces of crystalline form F before and after exposure to 25° C./60% relative humidity (RH) and 40° C./75% RH (open vial) after seven days.

FIG. 46 is an overlay of DSC traces of crystalline hydrogen sulfate form A before and after exposure to 25° C./60% relative humidity (RH) and 40° C./75% RH (open and closed vial) after fourteen days.

FIG. 47 is an overlay of DSC traces of crystalline form F before and after exposure to 25° C./60% relative humidity (RH) and 40° C./75% RH (open vial) after fourteen days.

FIG. 48 is an overlay of DSC traces of crystalline hydrogen sulfate form A before and after exposure to 25° C./60% relative humidity (RH) and 40° C./75% RH (open and closed vial) after four weeks.

FIG. 49 is an overlay of DSC traces of crystalline form F before and after exposure to 25° C./60% relative humidity (RH) (open vial) and 40° C./75% RH (open and closed vial) after four weeks.

FIG. 50 is an overlay of TG traces of crystalline hydrogen sulfate form A before and after exposure to 25° C./60% relative humidity (RH) and 40° C./75% RH (open and closed vial) after seven days.

FIG. 51 is an overlay of TG traces of crystalline form F before and after exposure to 25° C./60% relative humidity (RH) and 40° C./75% RH (open vial) after seven days.

FIG. 52 is an overlay of TG traces of crystalline hydrogen sulfate form A before and after exposure to 25° C./60% relative humidity (RH) and 40° C./75% RH (open and closed vial) after fourteen days.

FIG. 53 is an overlay of TG traces of crystalline form F before and after exposure to 25° C./60% relative humidity (RH) and 40° C./75% RH (open vial) after fourteen days.

FIG. 54 is an overlay of TG traces of crystalline hydrogen sulfate form A before and after exposure to 25° C./60% relative humidity (RH) and 40° C./75% RH (open and closed vial) after four weeks.

FIG. 55 is an overlay of TG traces of crystalline form F before and after exposure to 25° C./60% relative humidity (RH) and 40° C./75% RH (open vial) after four weeks.

FIG. 56 is an overlay of XRPD diffractograms of solid states of the free form of the compound of formula (I). Crystalline form M is a free form (top diffractogram). Crystalline form L is a mono-methanol solvate (second diffractogram from the top) of the compound of formula (I). Crystalline form K is a desolvated dichloromethane solvate (third diffractogram from the top) of the compound of formula (I). Crystalline form J is a free form of the compound of formula (I) (fourth diffractogram from the top). Crystalline form I is a mixture of crystalline forms B and M. Crystalline form H is a solvated/desolvated form of the compound of formula (I). Crystalline form G is a dichloromethane solvate of the compound of formula (I). Crystalline form F is a free form of the compound of formula (I). Crystalline form E is a 2-methyl tetrahydrofuran (2-MeTHF) solvate of the compound of formula (I). Crystalline form D is a mono-tetrahydrofuran solvate of the compound of formula (I). Crystalline form C is a free form of the compound of formula (I). Crystalline form B is a non-solvated form of the compound of formula (I). Crystalline form A is a mixture of crystalline form B and a solvate of the compound of formula (I).

FIG. 57 is an overlay of XRPD diffractograms of selected salts from a salt screen and a salt scale up. ML-1 is the first crystalline form of the maleic acid salt of the compound of formula (1) (top diffractogram). SA-2 is crystalline hydrogen sulfate form A (second diffractogram from the top). HCl-5, HCl-3, and HCl-1 are crystalline forms of the hydrochloride salt of the compound of formula (1) (third and fourth diffractograms from the top and bottom diffractogram, respectively).

FIG. 58 is a microscopic image of the crystalline hemi-sulfate form of the compound of formula (I) (crystalline sulfate form A) isolated from ethanol and sulfuric acid (0.55 equiv). The image was made in polarized, visible light.

FIG. 59 is an XRPD diffractogram of the crystalline hemi-sulfate form of the compound of formula (1) (crystalline sulfate form A).

FIG. 60 is an overlay of TG (top) and DSC (bottom) traces for the crystalline hemi-sulfate form of the compound of formula (I) (crystalline sulfate form A).

FIGS. 61A and 61B are TG and DSC traces for the crystalline hemi-sulfate form of the compound of formula (I) (crystalline sulfate form A) isolated from a scale-up experiment (see Example 2). FIG. 61A is an overlay of TG (top) and DSC (bottom) traces for crystalline sulfate form A that was isolated from the scale-up experiment. FIG. 61B is a DSC trace of the scaled-up crystalline sulfate form A that shows two overlapping endotherms.

FIG. 62 is a ¹H NMR spectrum (DMSO-de) of the crystalline hemi-sulfate form of the compound of formula (I) (crystalline sulfate form A).

FIG. 63 is an overlay of XRPD diffractograms from a 7-day stability assessment of crystalline sulfate form A. Diffractogram (1) is a reference of crystalline sulfate form A before humidity exposure (dry). Diffractogram (2) is of crystalline sulfate form A+another crystalline form of a sulfate salt of the compound of formula (I) after 7-day exposure to 75% RH at 40° C.

FIG. 64 is an XRPD diffractogram of crystalline form M of the compound of formula (I).

FIG. 65 is a DSC trace of crystalline form M of the compound of formula (I).

### DETAILED DESCRIPTION OF THE INVENTION

In general, the invention provides crystalline forms of a compound of formula (I) as well as its hydrogen sulfate, hemi-sulfate, and sulfate salts. The compound of formula (I) is of the following structure:

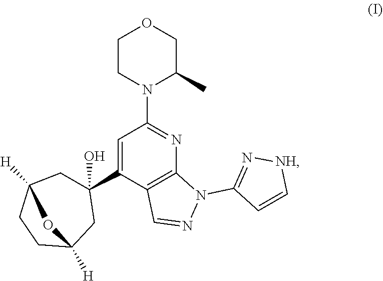

The crystalline form of the compound of formula (I) may be, e.g., crystalline form B, crystalline form C, crystalline form F, crystalline form M. The crystalline form of the hydrogen sulfate salt of the compound of formula (I) may be, e.g., crystalline hydrogen sulfate form A. The crystalline form of the hydrate of the hydrogen sulfate salt of the compound of formula (I) may be, e.g., crystalline hydrogen sulfate form B. The crystalline form of the hemi-sulfate salt of the compound of formula (I) may be, e.g., crystalline sulfate form A.

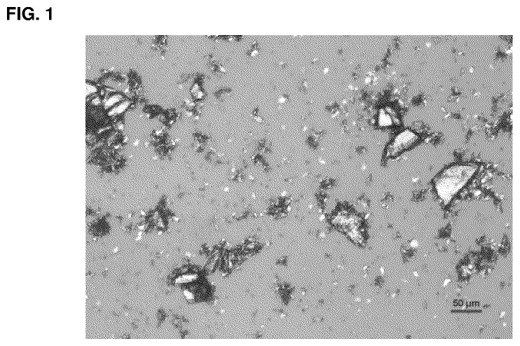

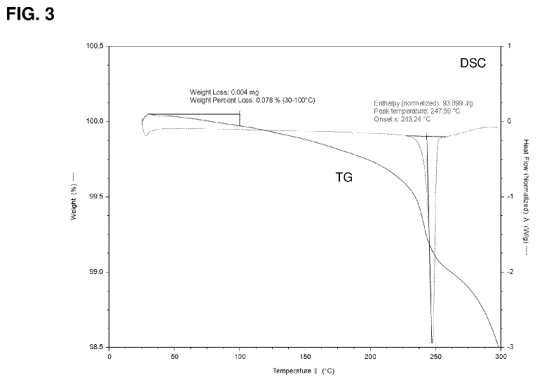

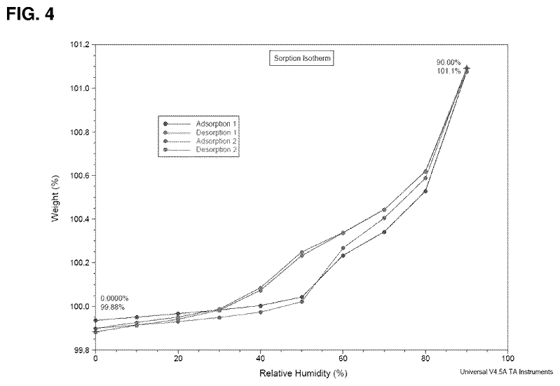

As described in the examples, crystalline form B was found to be crystalline with block-like crystals (see FIG. 1). The form had a melting range of 243° C. (onset) to 248° C. (peak) by DSC (see FIG. 3), and DVS analysis showed that it was slightly hygroscopic when subjected to 25° C./0-90% RH change (FIG. 4). The crystal form did not change according to XRPD analysis of the sample collected after DVS.

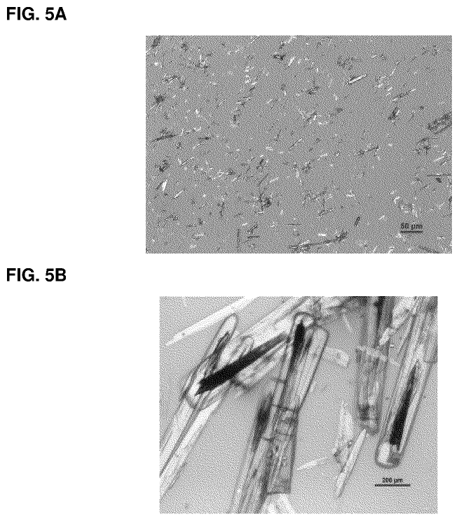

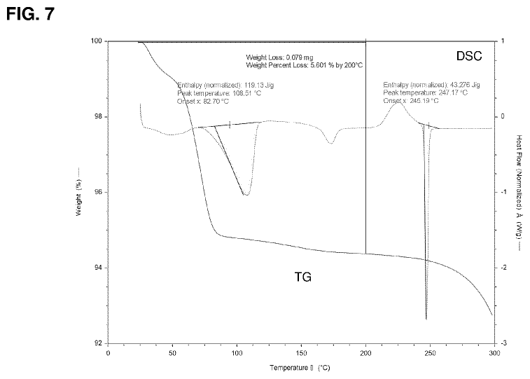

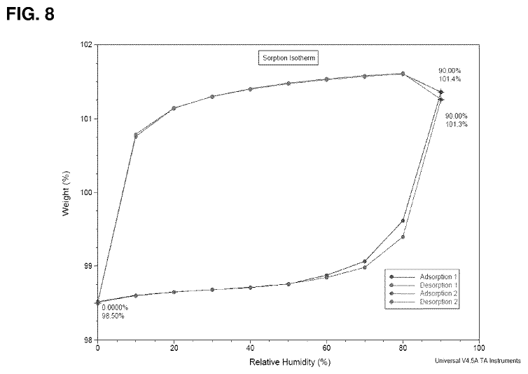

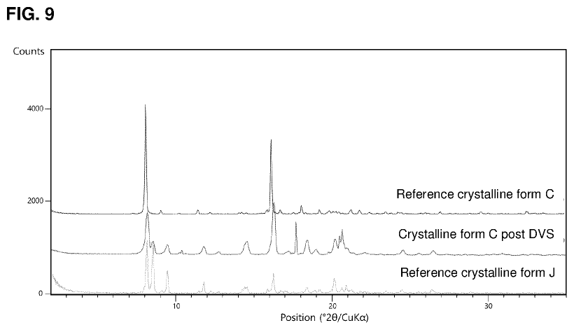

Crystalline form C was crystalline with needle/plate-like morphology (FIGS. 5A and 5B). Crystalline form C appeared to lose about 5.6% of its mass when heated to 200° C. by TG (FIG. 7). Crystalline form C melted at about 245° C. (onset) to 247° C. (peak) by DSC (FIG. 7). The material was slightly hygroscopic when subjected to 25° C./0-90% RH change (FIG. 8), however the XRPD pattern changed after DVS compared to the starting material and matched with crystalline form J, a free-base of the compound of formula (I) (FIG. 9).

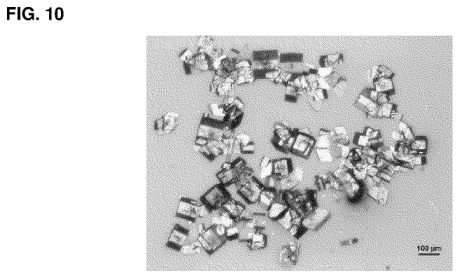

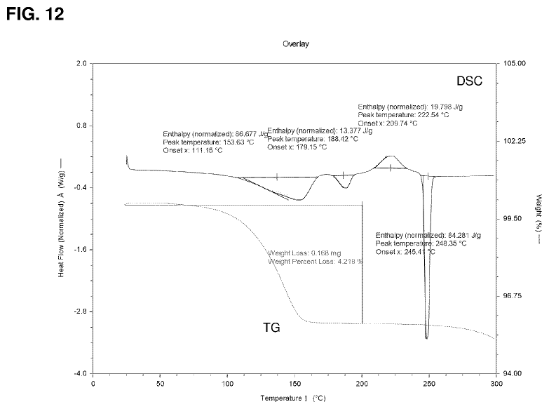

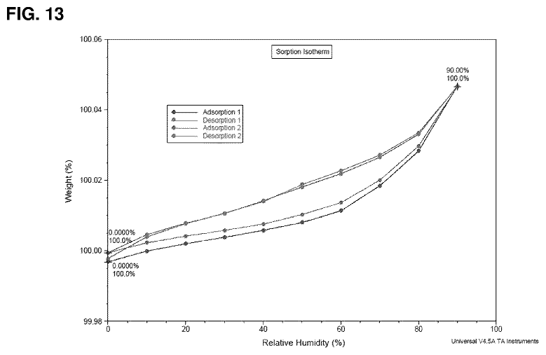

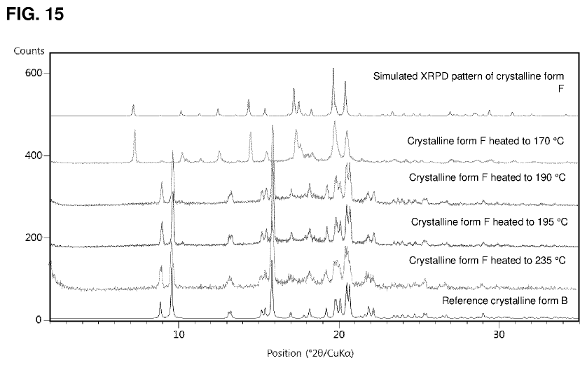

Crystalline form F was found to be crystalline with cube-like morphology (see FIG. 10). The form showed a weight loss of 4.2% when heated to 200° C. by TG (see FIG. 12). The first endotherm in DSC corresponded to dehydration and melting was observed at about 245° C. (onset) to about 248° C. (peak) (FIG. 12). The material was non hygroscopic by DVS (FIG. 13), and no form change was observed after heating/drying (FIG. 15) or exposure to 0-90% RH by XRPD analysis.

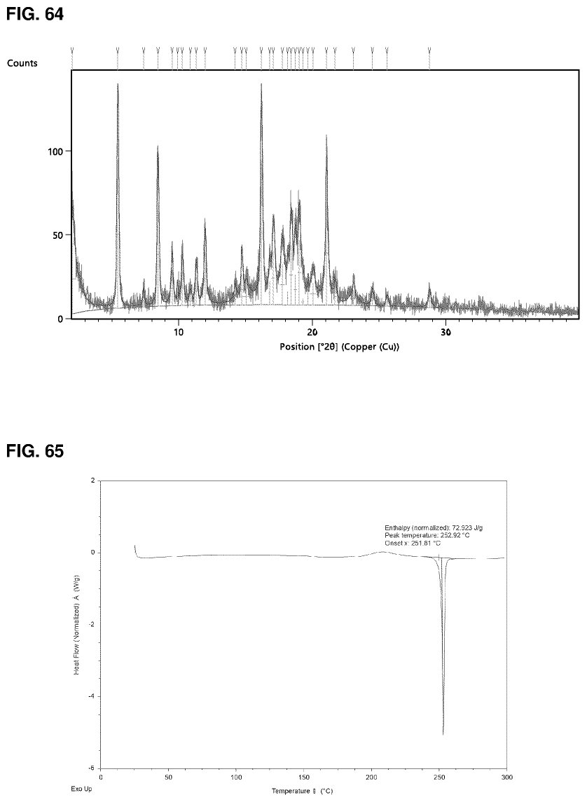

Crystalline form M was found to be crystalline (by XRPD, FIG. 64) with an endotherm in DSC corresponding to melting at about 252° C. (onset) to 253° C. (peak) (FIG. 65).

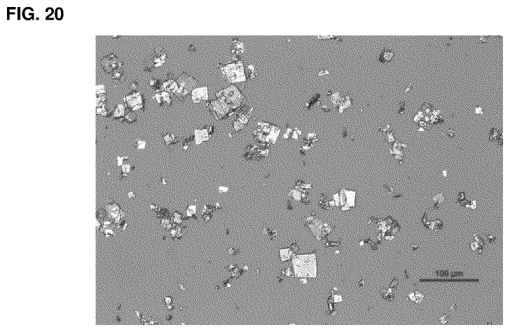

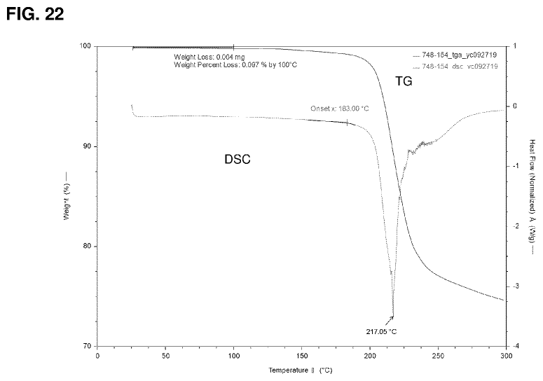

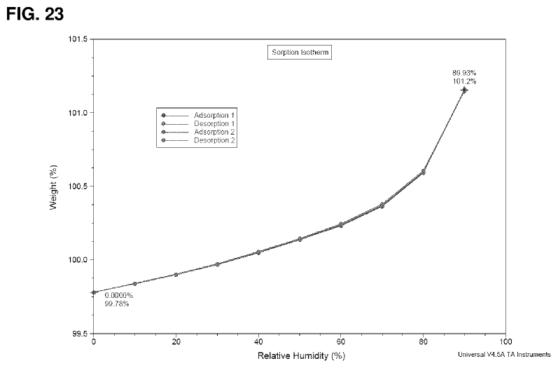

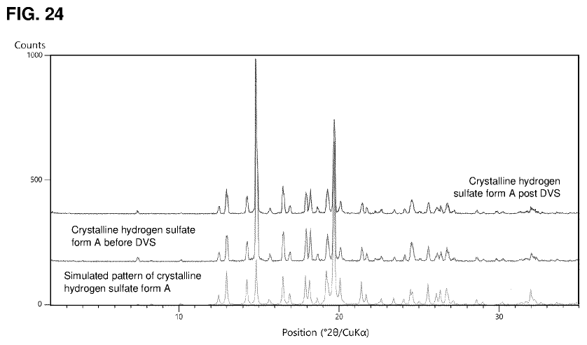

Crystalline hydrogen sulfate form A was found to be crystalline with square-like morphology (FIG. 20). The form had a weight loss of 0.097% when heated to 100° C. by TG, and melted at about 183° C. (onset) followed by degradation (FIG. 22). The material was slightly hygroscopic, with a moisture uptake of 1.42% when subjected to 0-90% RH change by DVS (FIG. 23). No form change was observed by XRPD analysis after subjecting the form to 0-90% RH (FIG. 24).

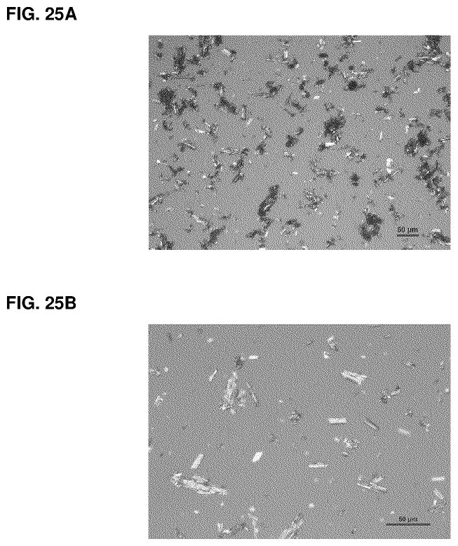

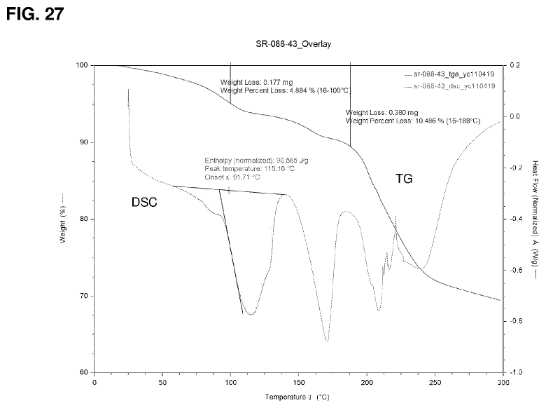

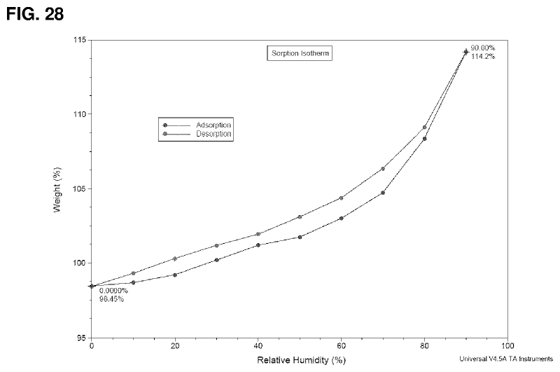

Crystalline hydrogen sulfate form B was found to be crystalline with needle/thin plate-like morphology (FIGS. 25A and 25B). The form had a weight loss of 10.5% when heated from 16° C. to 188° C. by TG (FIG. 27). The form appeared to lose water when heated to about 92° C. (onset) (FIG. 27). The material was hygroscopic, with a moisture uptake of 15.75% when subjected to 0-90% RH change by DVS (FIG. 28).

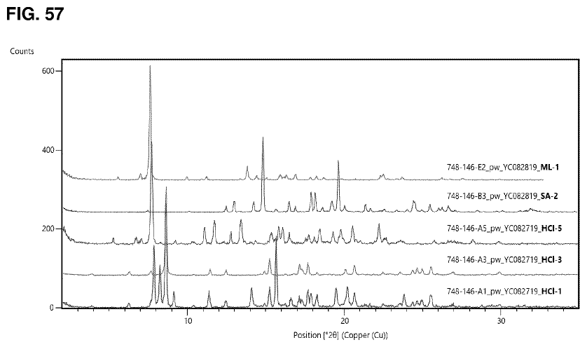

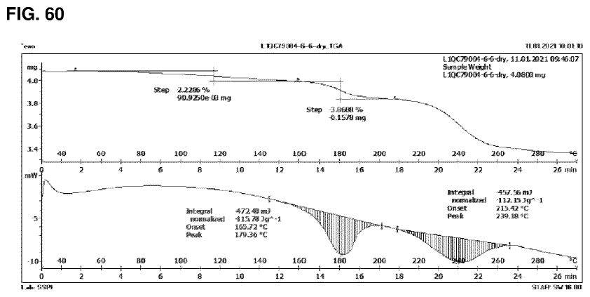

Crystalline sulfate form A was found to be crystalline with elongated particles (FIG. 58). The form had a weight loss of 2.2% when heated from 50° C. to 160° C., and a weight loss of 3.9% when heated from 160° C. to 210° C. by TG (FIG. 60, top). The first endotherm in DSC was observed at about 166° C. (onset) to about 179° C. (peak) (FIG. 60, bottom). The second endotherm in DSC was observed at about 215° C. (onset) to about 239° C. (peak) (FIG. 60, bottom).

The invention also provides a hydrogen sulfate salt of a compound of formula (I) or a hydrate thereof. The invention further provides a hemi-sulfate salt of a compound of formula (I). The invention further provides a sulfate salt of a compound of formula (I). Advantageously, the salts described herein (e.g., the hydrogen sulfate salt) may possess superior physicochemical properties for the purpose of pharmaceutical development.

### Methods of Use

Methods of treating a disease or condition having the symptom of cell hyperproliferation (e.g., a cancer) and methods for ATR kinase have been described in WO 2020/087170, the disclosure of which is incorporated by reference herein in its entirety.

A method of inhibiting ATR kinase in a cell (e.g., a cell in a subject) expressing ATR kinase may include contacting the cell with an effective amount of a crystalline form or a pharmaceutical composition containing the crystalline form disclosed herein.

A method of treating a subject in need thereof (e.g., a subject suffering from, and is in need of a treatment for, a disease or condition having the symptom of cell hyperproliferation (e.g., a cancer (e.g., a carcinoma, sarcoma, adenocarcinoma, leukemia, or melanoma) and/or a pre-malignant condition)) may include administering to the subject an effective amount of a crystalline form or a pharmaceutical composition containing the crystalline form disclosed herein.

Non-limiting examples of carcinomas include medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, Schneiderian carcinoma, scirrhous carcinoma, carcinoma scroti, signet ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

In some embodiments, the crystalline form used in the methods described herein is a crystalline form B. In some embodiments, the crystalline form used in the methods described herein is a crystalline form C. In some embodiments, the crystalline form used in the methods described herein is a crystalline form F. In some embodiments, the crystalline form used in the methods described herein is a crystalline form M. In some embodiments, the crystalline form used in the methods described herein is a crystalline hydrogen sulfate form A. In some embodiments, the crystalline form used in the methods described herein is a crystalline hydrogen sulfate form B. In some embodiments, the crystalline form used in the methods described herein is a crystalline sulfate form A.

Non-limiting examples of leukemias include nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia. In some embodiments, the crystalline form used in the methods described herein is a crystalline form B. In some embodiments, the crystalline form used in the methods described herein is a crystalline form C. In some embodiments, the crystalline form used in the methods described herein is a crystalline form F. In some embodiments, the crystalline form used in the methods described herein is a crystalline form M. In some embodiments, the crystalline form used in the methods described herein is a crystalline hydrogen sulfate form A. In some embodiments, the crystalline form used in the methods described herein is a crystalline hydrogen sulfate form B. In some embodiments, the crystalline form used in the methods described herein is a crystalline sulfate form A.

Non-limiting examples of melanomas include acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma. In some embodiments, the crystalline form used in the methods described herein is a crystalline form B. In some embodiments, the crystalline form used in the methods described herein is a crystalline form C. In some embodiments, the crystalline form used in the methods described herein is a crystalline form F. In some embodiments, the crystalline form used in the methods described herein is a crystalline form M. In some embodiments, the crystalline form used in the methods described herein is a crystalline hydrogen sulfate form A. In some embodiments, the crystalline form used in the methods described herein is a crystalline hydrogen sulfate form B. In some embodiments, the crystalline form used in the methods described herein is a crystalline sulfate form A.

Non-limiting examples of cancer include prostate cancer, thyroid cancer, endocrine system cancer, brain cancer, breast cancer, cervix cancer, colon cancer, head & neck cancer, liver cancer, kidney cancer, lung cancer, non-small cell lung cancer, melanoma, mesothelioma, ovarian cancer, sarcoma, stomach cancer, uterus cancer, medulloblastoma, ampullary cancer, colorectal cancer, or pancreatic cancer. In some embodiments, the crystalline form used in the methods described herein is a crystalline form B. In some embodiments, the crystalline form used in the methods described herein is a crystalline form C. In some embodiments, the crystalline form used in the methods described herein is a crystalline form F. In some embodiments, the crystalline form used in the methods described herein is a crystalline form M. In some embodiments, the crystalline form used in the methods described herein is a crystalline hydrogen sulfate form A. In some embodiments, the crystalline form used in the methods described herein is a crystalline hydrogen sulfate form B. In some embodiments, the crystalline form used in the methods described herein is a crystalline sulfate form A.

Other non-limiting examples of cancer include Hodgkin's Disease, Non-Hodgkin's Lymphoma, multiple myeloma, neuroblastoma, glioma, glioblastoma multiforme, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphoma, esophageal cancer, genitourinary tract cancer, malignant hypercalcemia, endometrial cancer, adrenal cortical cancer, neoplasms of the endocrine or exocrine pancreas, medullary thyroid cancer, or papillary thyroid cancer. In some embodiments, the crystalline form used in the methods described herein is a crystalline form B. In some embodiments, the crystalline form used in the methods described herein is a crystalline form C. In some embodiments, the crystalline form used in the methods described herein is a crystalline form F. In some embodiments, the crystalline form used in the methods described herein is a crystalline form M. In some embodiments, the crystalline form used in the methods described herein is a crystalline hydrogen sulfate form A. In some embodiments, the crystalline form used in the methods described herein is a crystalline hydrogen sulfate form B. In some embodiments, the crystalline form used in the methods described herein is a crystalline sulfate form A.

### Pharmaceutical Compositions

A crystalline form described herein (e.g., a crystalline form of a compound of formula (I)) may be formulated into a pharmaceutical composition for administration to human subjects in a biologically compatible form suitable for administration in vivo. A pharmaceutical composition typically includes an active agent as described herein and a physiologically acceptable excipient (e.g., a pharmaceutically acceptable excipient). Formulation principles for the compound of formula (I) have been described in WO 2020/087170, the disclosure of which is incorporated by reference herein in its entirety. The crystalline forms described herein are especially beneficial for solid pharmaceutical compositions, e.g., solid dosage forms (e.g., tablets, powders, lozenges, sachets, cachets, and soft and hard gelatin capsules).

The compound of formula (I) may be administered, for example, by oral, parenteral, buccal, sublingual, nasal, or rectal administration, and the pharmaceutical compositions formulated accordingly. Preferably, a crystalline form of the compound of formula (I) is administered orally.

Suitable pharmaceutical carriers, as well as pharmaceutical necessities for use in pharmaceutical formulations, are described in Remington: The Science and Practice of Pharmacy, 21^{st} Ed., Gennaro, Ed., Lippencott Williams & Wilkins (2005), a well-known reference text in this field, and in the USP/NF (United States Pharmacopeia and the National Formulary).

### EXAMPLES

The following examples are meant to illustrate the invention. They are not meant to limit the invention in anyway.

Abbreviations Explanations
MeCN Acetonitrile
n-BuOH 1-Butanol
2-BuOH 2-Butanol
t-BuOH tert-Butanol/2-Methylpropan-2-o/
CPME Cyclopentyl methyl ether
DCM Dichloromethane
DMAc Dimethylacetamide
DME Dimethoxyethane
DMF N,N-Dimethylformamide
DMSO Dimethyl sulfoxide
DSC Differential Scanning Calorimetry
DVS Dynamic Vapor Sorption
EtOAc Ethyl Acetate
EtOH Ethanol
FB Free Base
¹H NMR Proton Nuclear Magnetic Resonance
HPLC High Performance Liquid Chromatography
IPA Isopropanol/Propan-2-ol
IPAc Isopropyl Acetate
mDSC Modulated Differential Scanning Calorimetry
MC Methyl Cellulose
MEK Butanone/Methyl Ethyl Ketone
MeOH Methanol
2-MeTHF 2-Methyl Tetrahydrofuran
MIBK Methyl Isobutyl Ketone
NMP N-Methyl-2-pyrrolidone
NPA 1-Propanol/n-Propanol
PLM Polarized Light Microscopy
XRPD X-Ray Powder Diffraction
RH Relative Humidity
RT Room Temperature (20-25° C.)
SLS Sodium Lauryl Sulfate
TG Thermogravimetry
THE Tetrahydrofuran
SCXRD Single Crystal X-Ray Diffraction

Methods. During the preparations described in the examples, the methods described herein below have been used to monitor the experimental setups and products obtained.

### X-Ray Powder Diffraction (XRPD)

### Bruker D2 Phaser 2nd Gen

Instrument: Bruker D2 Phaser 2nd Gen
Parameters: X-Ray tube Cu (Kα) with 1.54184 [A]; Tube Voltage 30 kV; Tube current 10 mA
Scanning range: 2 to 4°2θ (degree)
Step size: 0.01°
Scanning speed: 1 or 2° per minute

Malvern Panalytical
Instrument: Panalytical X′Pert3 Powder
Parameters: X-Ray tube Cu (Kα); tube voltage 45 kV; tube current 40 mA
Scanning range: 2 to 4°2θ (degree)
Step size: 0.01°
Scanning speed: 6° per minute
   Peak lists were generated using HighScore Plus:
Parameters: Minimum significance: 2.00
   Minimum tip width Gonio: 0.01
   Maximum tip width Gonio: 1.00
   Peak base width Gonio: 2.00
Method: Minimum 2^{nd} derivative
   Peaks were evaluated visually and removed/added manually. Profile fitting with default setting was used.

### Nuclear Magnetic Resonance (NMR)

Instrument: Bruker 400 Ultrashield
Solvent: DMSO

### Thermogravimetry (TG)

Instrument: TA Instruments Discovery TGA
Parameters: Ramp 10° C. per minute, 25 to 300° C., 50 mL/min N₂ sweep

### Dynamic Vapor Sorption Analysis (DVS)

Instrument: DVS Intrinsic, Surface Measurement Systems
Parameters: 25° C., 0-90-0% RH for 2 cycles

### Polarized Light Microscopy (PLM)

Instrument: Nikon Eclipse Ci POL
Camera: Nikon DS-Fi3
Software: Nikon NIS Elements

### Differential Scanning Calorimetry (DSC)

Instrument: TA Instruments Discovery DSC
Parameters: Ramp 5/10/20° C. per minute, up to 300° C.

### Example 1. Preparation of the Crystalline Forms of the Compound of Formula (I)

Preparation of the compound of formula (I) is described in WO 2020/087170, the disclosure of which is incorporated by reference herein in its entirety.

Preparation of the compound of formula (I) as crystalline form B. Crystalline form B of the compound of formula (I) was obtained by suspending the compound of formula (I) (50 mg, crystalline form A) in EtOAc, then adding methanol to dissolve. The solvent was evaporated to obtain crystals of crystalline form B. Table 1 summarizes the physical and chemical characteristics for crystalline form B.

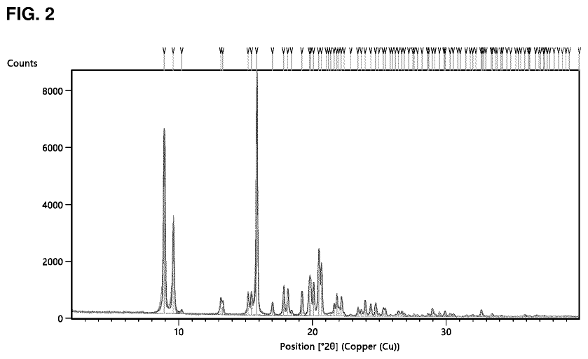

TABLE 1
Physical Parameters

| | |
|---|---|
| Appearance | White powder (block-like crystals under |
| | microscope) (FIG. 1) |
| Thermogravimetry | Negligible weight loss from 30° C. to 100° C. |
| (TG) | (FIG. 3) |
| Differential | Melting at 243° C. (onset) to 248° C. (peak), with |
| Scanning | enthalpy about 93.9 J/g (FIG. 3) |
| Calorimetry (DSC) | |
| Hygroscopicity | Slightly hygroscopic (e.g., ≥0.2% and <2% |
| | weight increase at 80% RH, 25° C.) (FIG. 4) |
| Powder X-ray | Crystalline (FIG. 2) |
| Diffraction | |

### Preparation of the Compound of Formula (I) as Crystalline Form C.

The compound of formula (I) (crystalline form A) was suspended in acetone-water, and after evaporation, crystalline form C was obtained after vacuum drying at room temperature overnight. Crystalline form C was also isolated from aqueous ethanol. The physical and chemical characteristics of crystalline form C are summarized in Table 2.

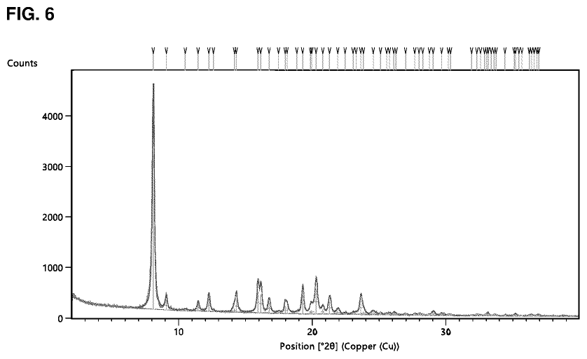

TABLE 2
Physical Parameters

| | |
|---|---|
| Appearance | White powder (needle/plate-like crystals |
| | under microscope) (FIGS. 5A and 5B) |
| Melting Point/Range | 245° C. (onset) to 247° C. (peak) by DSC (FIG. 7) |
| Thermogravimetry | 5.6% weight loss from 25° C. to 200° C. (FIG. 7) |
| (TG) | |
| Differential | First endotherm at 83° C. (onset) to 109° C. (peak) |
| Scanning | (FIG. 7) |
| Calorimetry (DSC) | |
| Hygroscopicity | Slightly hygroscopic (e.g., ≥0.2% and <2% weight |
| | increase at 80% RH, 25° C.) (FIG. 8) |
| Powder X-ray | Crystalline (FIG. 6) |
| Diffraction | |

### Preparation of the Compound of Formula (I) as Crystalline Form F.

The compound of formula (I) (crystalline form A) was suspended in aqueous ethanol at room temperature, which afforded large, long rod/prism crystals of crystalline form C which later converted to cube-like crystals of crystalline form F shown in FIG. 10, and was confirmed by comparison against the simulated XRPD from the SCXRD. Bulk crystals of crystalline form F were oven-dried and analyzed by XRPD, DSC, TG and DVS. Table 3 summarizes the physical and chemical characteristics of crystalline form F.

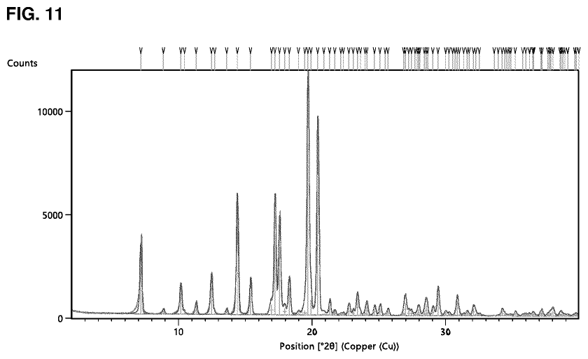

TABLE 3
Physical Parameters

| | |
|---|---|
| Appearance | White powder (cube-like crystals under |
| | microscope) (FIG. 10) |
| Melting Point/Range | 245° C. (onset) to 248° C. (peak) by DSC (FIG. 12) |
| Thermogravimetry | 4.2% weight loss from 25° C. to 200° C. (FIG. 12) |
| (TG) | |
| Differential | First endotherm at 111° C. (onset) to 154° C. |
| Scanning | (peak) (FIG. 12) |
| Calorimetry (DSC) | |
| Hygroscopicity | Non hygroscopic (e.g., <0.2% weight increase at |
| | 80% RH, 25° C.) (FIG. 13) |
| Powder X-ray | Crystalline (FIG. 11) |
| Diffraction | |

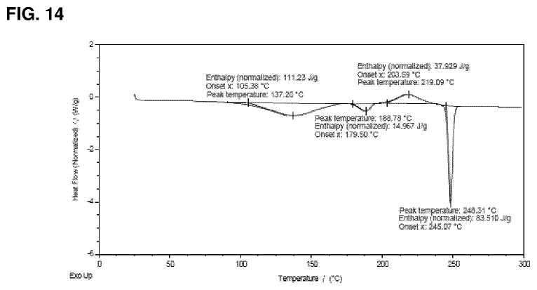

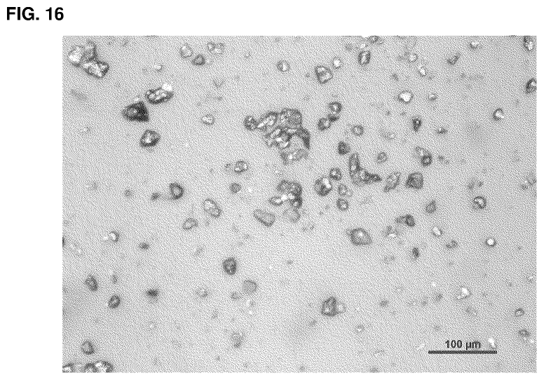

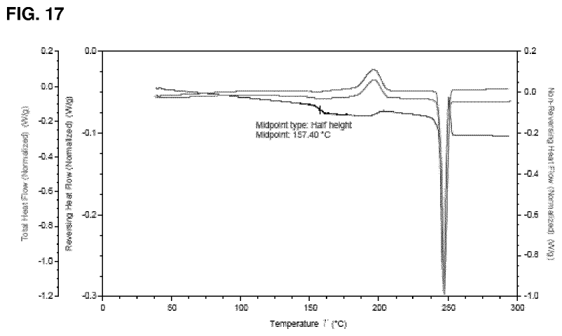

Furthermore, to better understand behaviors of crystalline form F upon heating, samples were generated by heating crystalline form F to 170° C., 190° C., and 195° C., using a TG instrument, and were analyzed by XRPD. Prior to selection of these temperature points, an open-pan DSC was run (FIG. 14). The results of the XRPD analysis are shown in FIG. 15, together with the pattern collected previously on a sample heated to 235° C. It showed that 1) the dehydrated/partially dehydrated phase (after heated to 170° C.) displayed the same pattern as crystalline form F; 2) crystallization of the neat form (crystalline form B) from melt of the dehydrated phase was observed by XRPD analysis as early as at 190° C. while the onset of the exotherm (crystallization) was at 203° C.; 3) the endotherm at about 245° C. (onset) was melting of the neat form (crystalline form B). XRPD analysis of these samples showed reduced crystallinity. PLM of the sample heated to 195° C. appeared to contain some amorphous particles (FIG. 16). To confirm the amorphous particles, the sample was analyzed by modulated DSC. Glass transition at 157° C. (midpoint) was observed (FIG. 17).

### Preparation of Other Crystalline Forms of the Compound of Formula (I).

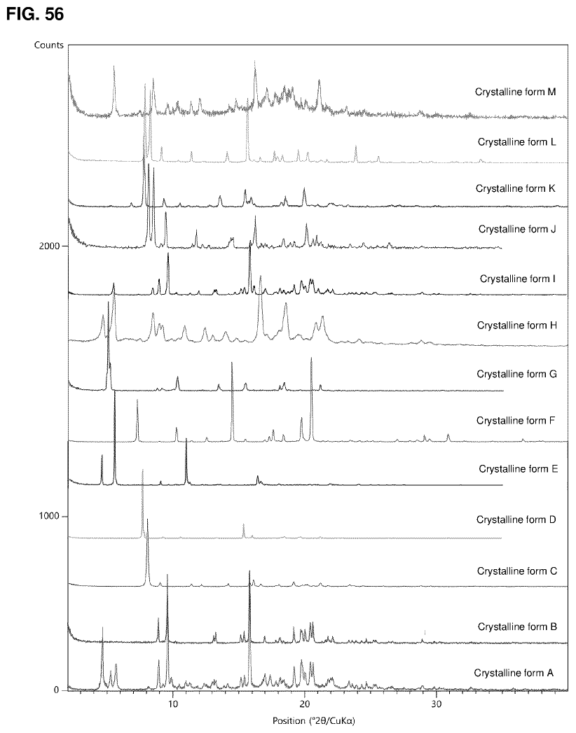

Table 4 summarizes the crystalline free base forms. FIG. 56 shows an overlay of the respective XRPD patterns of each solid state.

TABLE 4

| Pattern | Solid State |
|---|---|
| Crystalline form A | Likely mixture of crystalline form B and |
| | solvate/desolvated solvate |
| Crystalline form B | Free form, neat (a non-solvated form) |
| Crystalline form C | Free form |
| Crystalline form D | Mono-THF solvate |
| Crystalline form E | 2-MeTHF solvate |
| Crystalline form F | Free form |
| Crystalline form G | DCM solvate |
| Crystalline form H | Solvated/desolvated form |
| Crystalline form I | Crystalline form B and form M, mixture of |
| | non-solvated forms |
| Crystalline form J | Free form |
| Crystalline form K | Desolvated DCM solvate |
| Crystalline form L | Mono-MeOH solvate |
| Crystalline form M | Second Neat Form (a non-solvated form) |

Table 5 summarizes other solvents from which various crystalline forms were isolated.

TABLE 5

| | Compound | Solvent | | | | |
|---|---|---|---|---|---|---|
| | of Formula | Volume | XRPD | After 24 | After 3 | After oven-drying |
| Solvent | (I) (mg) | (mL) | Pattern | hrs | days | over weekend |
| Acetone | 50.6 | 0.9 | Crystalline | Crystalline | Crystalline | Crystalline |
| | | | form B | form D | form D | form K |
| THF | 50.6 | 0.5 | Crystalline | Crystalline | Crystalline | Crystalline |
| | | | form D | form D | form D | form D′ |
| 2-MeTHF | 50.2 | 0.5 | Crystalline | Crystalline | Crystalline | Crystalline |
| | | | form E | form E | form E | form E′ |
| Water | 50.0 | 0.5 | Crystalline | Crystalline | Crystalline | Crystalline |
| | | | form F | form C | form C | form J |
| DCM | 50.4 | 0.9 | Crystalline | Crystalline | Crystalline | Crystalline |
| | | | form G | form K | form K | form K |

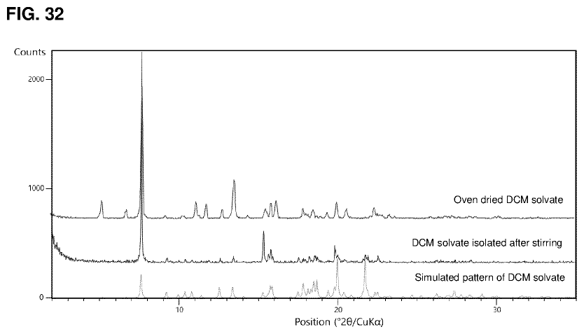

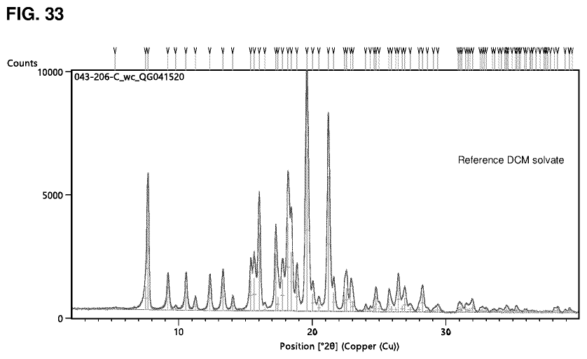

Crystalline form K was obtained after suspending 50 mg of the compound of formula (I) (crystalline form A) in DCM for 3 days. The sample was oven dried overnight and XRPD was collected on the solid. The material was a DCM solvate of the free-base compound before oven drying, and confirmed by SCXRD. The XRPD changed after oven drying at 48° C. (FIG. 32).

Crystalline form D was obtained after suspending 50 mg of the compound of formula (I) (crystalline form A) in THF for 3 days. The sample was dried overnight and XRPD was collected on the solid (FIG. 33). The material was a mono-THF solvate of the free-base compound.

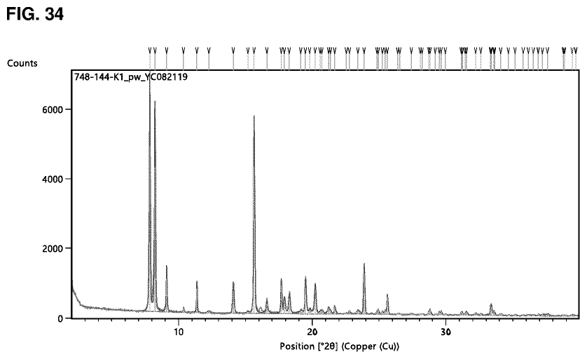

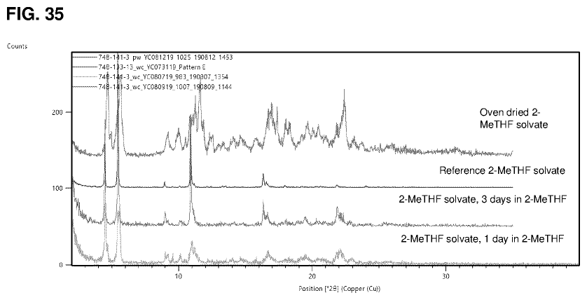

Crystalline form E was obtained after suspending 50 mg of the compound of formula (I) (crystalline form A) in 2-MeTHF for 3 days. The sample was dried overnight and XRPD was collected on the solid (FIG. 34). The material was a 2-MeTHF solvate of the free-base compound, which was produced by stirring the compound of formula (I) in 2-MeTHF, overnight (bottom trace in FIG. 35), followed by stirring at room temperature for 2 days (second-from-the-bottom trace in FIG. 35). The XRPD changed after oven drying at 48° C. overnight (top trace in FIG. 35).

Crystalline form M was obtained after heating crystalline form H (which was isolated from isopropyl acetate) to 248° C.

### Example 2. Preparation of the Crystalline Hydrogen Sulfate Forms of the Compound of Formula (I)

Preparation of crystalline hydrogen sulfate form A. Crystalline hydrogen sulfate form A (neat) was prepared using crystalline form H (82 mg) in EtOH. Sulfuric acid was added to the suspension at 1:1.5 (compound of formula (1):H₂SO₄) molar ratio, and precipitate was observed. After 18 hours of stirring, the solids were isolated and oven vacuum dried at room temperature. Table 6 summarizes the physical and chemical characteristics for crystalline hydrogen sulfate form A.

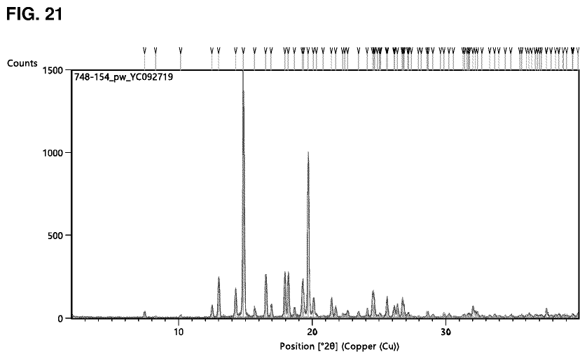

TABLE 6
Physical Parameters

| | |
|---|---|
| Appearance | White powder (square-like crystals under |
| | microscope) (FIG. 20) |
| Melting Point/Range | 183° C. (onset) followed by degradation by |
| | DSC (FIG. 22) |
| Thermogravimetric | 0.097% weight loss from 26° C. to 100° C. |
| Analysis (TG) | (FIG. 22) |
| Differential | Melting at 183° C. (onset) to 217° C. (peak), |
| Scanning | followed by degradation (FIG. 22) |
| Calorimetry (DSC) | |
| Hygroscopicity | Slightly hygroscopic (e.g., ≥0.2% and <2% weight |
| | increase at 80% RH, 25° C.) (FIG. 23) |
| Powder X-ray | Crystalline (FIG. 21) |
| Diffraction | |

The material was slightly hygroscopic, with a moisture uptake of 1.42% when subjected to 0-90% RH change by DVS (FIG. 23). No form change was observed by XRPD after subjecting the crystalline form to DVS (FIG. 24).

Preparation of crystalline hydrogen sulfate form B. Approximately 1.0 g of the compound of formula (I) (97.3% pure by quantitative NMR; 99.9% pure by HPLC (A); 0.9% water (w/w, as determined by Karl Fisher titration); 1.9% residual EtOAc (w/w)) was added to a 20 mL vial followed by the addition of 2 mL of THF:water (9:1 v/v). A white slurry formed and was left to stir at 45° C. for 1 h. A solution of concentrated sulfuric acid (95-98%) was then prepared in THF:water (9:1 v/v) with concentration of 98.0 mg/mL. A total of 3.1 equiv of sulfuric acid (total volume of 8.0 mL from prepared solution) was then added in eight equal portions, dropwise, over 2 h to the slurry of compound of formula (I). After each addition, the sample was left to stir for 5 min then the pH of the slurry was measured. The pH value became above saturation level of the pH probe after the first addition. The sample was seeded with crystalline hydrogen sulfate form B (spatula tip) after the addition of the 6^{th} portion. When all the required volume of sulfuric acid was added, the sample was left to stir at 45° C. for 1 h then at RT overnight. The observations from the scale-up experiment are included in Table 15. After overnight stirring, the sample was filtered for 3 min and washed with 2×1 vol. of THF:water (9:1 v/v). An aliquot was collected from the wet cake and XRPD analysis confirmed the solid state to be crystalline hydrogen sulfate form B. The filtered solids were then transferred to a vacuum oven and left to dry at 50° C. for 5 h under active vacuum (−29.5 inHg). The sample was then transferred to an oven at RT and left to dry under active vacuum for 5 h, then under static vacuum for 3 days. XRPD analysis confirmed that crystalline hydrogen sulfate form B was stable after drying. The yield after drying was 79.6%, w/w (with respect to the mono-sulfate mono-hydrate salt). The sulfate content determined for the sample was 19.5 wt %, which is slightly higher than the value of 18.6 wt % expected from a mono-sulfate mono-hydrate salt. The stoichiometry of crystalline hydrogen sulfate form B determined from the sulfate content was 1:1.05 API: mono-sulfate mono-hydrate. Table 7 summarizes the physical and chemical characteristics for crystalline hydrogen sulfate form B.

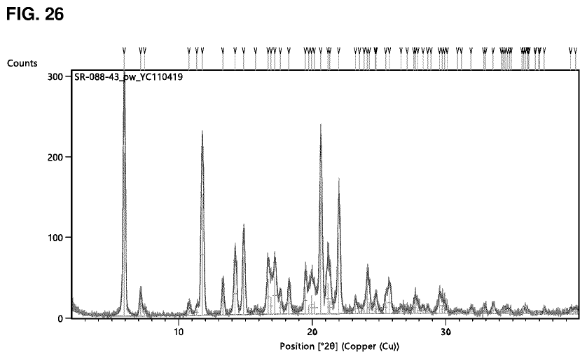

TABLE 7
Physical Parameters

| | |
|---|---|
| Appearance | White powder (needle/thin plate-like crystals |
| | under microscope) (FIGS. 25A and 25B) |
| Melting Point/Range | n/a |
| Thermogravimetry | 10.5% weight loss from 16° C. to 188° C. (FIG. |
| (TG) | 27) |
| Differential | First endotherm at 91.71° C. (onset), degrades |
| Scanning | above 188° C. (FIG. 27) |
| Calorimetry (DSC) | |
| Hygroscopicity | Hygroscopic (e.g., ≥2% and <15% weight increase |
| | at 80% RH, 25° C.) (FIG. 28) |
| Powder X-ray | Crystalline (FIG. 26) |
| Diffraction | |

The material was hygroscopic, with a moisture uptake of 15.75% when subjected to 0-90% RH change by DVS (FIG. 28).

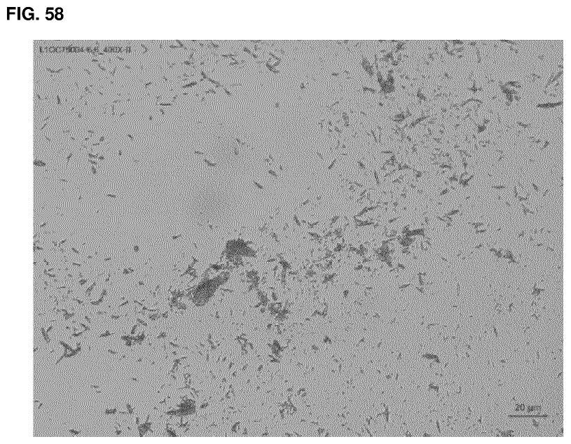

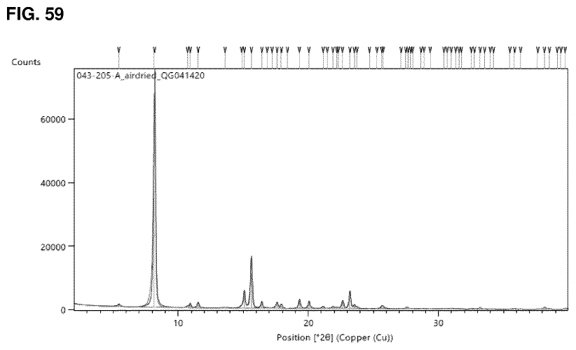

Preparation of crystalline sulfate form A. Crystalline sulfate form A was prepared from the compound of formula (I) stirred in ethanol with 0.55, 0.75, 1.0, and 1.5 equiv of sulfuric acid (see FIG. 59 for the XRPD pattern). The scale-up with 0.55 equiv proceeded as follows: approximately 1.0 g of (97.3% pure by quantitative NMR; 99.9% pure by HPLC (A); 0.9% water (w/w, as determined by Karl Fisher titration); 1.9% residual EtOAc (w/w)) was added to a 20 mL vial followed by the addition of 15 mL of EtOH. A thin, white slurry formed and was left to stir at 45° C. for 1.5 h. A solution of concentrated sulfuric acid (95-98%) was then prepared in EtOH with a concentration of 46.0 mg/mL. A total of 0.55 equiv of sulfuric acid (total volume of 3.0 mL from prepared solution) was then added in eight equal portions, dropwise, over 2 h to the slurry. The sample was seeded with crystalline sulfate form A (spatula tip) after the addition of the 4th portion. Aliquots were collected, filtered, and analyzed by XRPD after the addition of 4th (0.28 equiv) and the 5th (0.34 equiv) portions. When all the required volume of sulfuric acid was added, the sample was left to stir at 45° C. for 2 h then at RT for 45 min. An aliquot was collected at that time and crystalline sulfate form A was confirmed by XRPD analysis. The sample was then filtered for 3 min and washed with 2×1 vol. of EtOH. The filtered solids were then transferred to a vacuum oven and left to dry at 50° C. under static vacuum (−29.5 inHg) overnight (−16 h), then under active vacuum for 4 h. XRPD analysis confirmed that crystalline sulfate form A was stable after drying. The sulfate content of the sample (determined by ELTRA) was 10.74 wt %, consistent with the value of 9.64 wt % expected for hemi-sulfate. The stoichiometry of crystalline sulfate form A determined from the sulfate content was 1:0.56 API:sulfate.

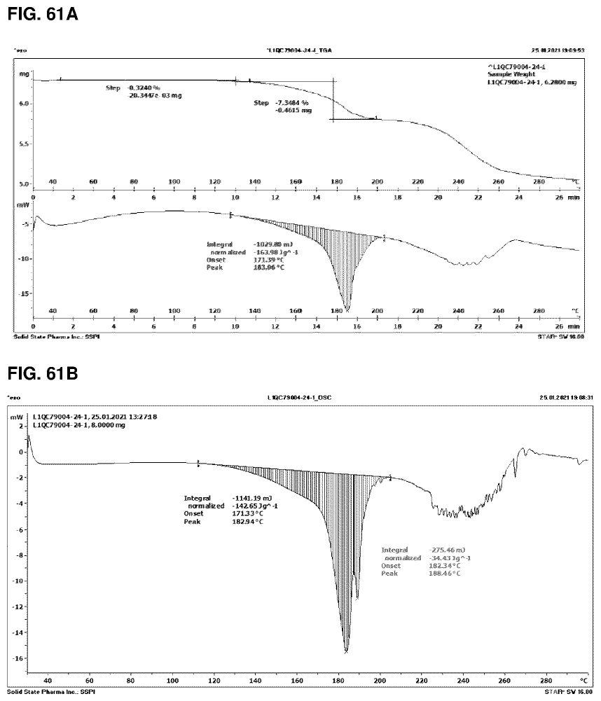

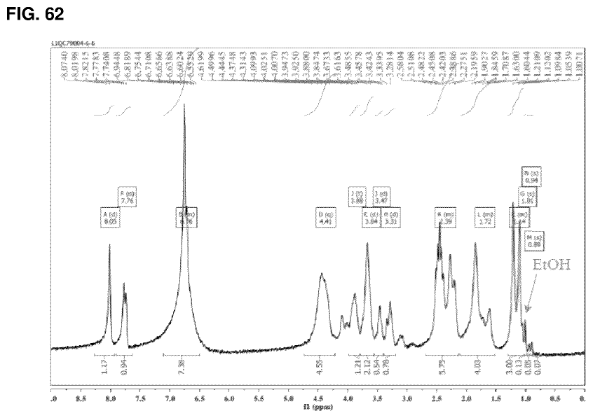

Crystalline sulfate form A showed slightly reduced crystallinity after drying under active vacuum (−29.5 inHg) for 3 h at 50° C. TG thermogram of crystalline sulfate form A showed two mass losses of 2.2 wt. % between 50 and 160° C., and 3.9 wt. % between 160 and 210° C. (FIG. 60, top). The DSC thermogram of crystalline sulfate form A showed two endothermic peaks with onsets at 165.7 and 215.4° C. (FIG. 60, bottom). TG thermogram of crystalline sulfate form A from the scale-up showed two mass losses of 0.32 wt % between 40 and 140° C., and 7.35 wt % between 140 and 200° C. (FIG. 61A, top). Two overlapping endotherms were observed from the DSC thermogram, with onset temperatures at 171.3 and 182.3° C. (FIG. 61A (bottom) and FIG. 61B). Microscopy images of crystalline sulfate form A showed elongated particles (FIG. 58). ¹H NMR spectrum of crystalline sulfate form A is shown in FIG. 62.

TABLE 7A
Physical Parameters

| | |
|---|---|
| Appearance | White solid (FIG. 58) |
| Melting Point/Range | About 171° C. (onset) to 183° C. (peak) by DSC |
| | (FIG. 61A) |
| Thermogravimetry | 0.32% weight loss from 40° C. to 140° C., and |
| (TG) | 7.35% weight loss from 140° C. to 200° C. (FIG. |
| | 61A, top) |
| Differential | First endotherm at 171.3° C. (onset), second |
| Scanning | endotherm 182.3° C. (FIG. 61A, bottom and FIG. |
| Calorimetry (DSC) | 61B) |
| Hygroscopicity | Hygroscopic (2.8% weight increase at 2-95% RH) |
| Powder X-ray | Crystalline (FIG. 59) |
| Diffraction | |

Example 3. Analysis of the Crystalline Forms of the Compound of Formula (I)

### X-Ray Powder Diffraction

The XRPD peak list for crystalline form B is provided in Table 8, the XRPD peak list for crystalline form C is provided in Table 9, the XRPD peak list for crystalline form F is provided in Table 10, the XRPD peak list for crystalline form M is provided in Table 10A, the XRPD peak list for crystalline hydrogen sulfate form A is provided in Table 11, the XRPD peak list for crystalline hydrogen sulfate form B is provided in Table 12, and the XRPD peak list for crystalline sulfate form A is provided in Table 12A. The XRPD diffractogram for crystalline form B is provided in FIG. 2, the XRPD diffractogram for crystalline form C is provided in FIG. 6, the XRPD diffractogram for crystalline form F is provided in FIG. 11, and the XRPD diffractogram for crystalline form M is provided in FIG. 64. The XRPD diffractogram for crystalline hydrogen sulfate form A is provided in FIG. 21, the XRPD diffractogram for crystalline hydrogen sulfate form B is provided in FIG. 26, and the XRPD diffractogram for crystalline sulfate form A is provided in FIG. 59.

TABLE 8

| Pos. | Height | FWHM Left | d-spacing | Rel. Int. |
|---|---|---|---|---|
| [°2θ] | [cts] | [°2θ] | [Å] | [%] |
| 8.9188 | 4422.94 | 0.1283 | 9.90709 | 73.63 |
| 9.5956 | 2162.27 | 0.1408 | 9.20979 | 35.99 |
| 10.2222 | 81.49 | 0.1190 | 8.64655 | 1.36 |
| 13.1483 | 398.16 | 0.1427 | 6.72813 | 6.63 |
| 13.2985 | 247.69 | 0.1248 | 6.65249 | 4.12 |
| 15.1737 | 410.21 | 0.1010 | 5.83434 | 6.83 |
| 15.4332 | 471.85 | 0.1962 | 5.73679 | 7.85 |
| 15.8346 | 6007.30 | 0.1136 | 5.59226 | 100.00 |
| 17.0030 | 304.96 | 0.1197 | 5.21051 | 5.08 |
| 17.8439 | 746.90 | 0.1093 | 4.96683 | 12.43 |
| 18.1555 | 647.74 | 0.1241 | 4.88228 | 10.78 |
| 18.4221 | 121.42 | 0.1201 | 4.81222 | 2.02 |
| 19.2052 | 629.03 | 0.1142 | 4.61772 | 10.47 |
| 19.7832 | 886.46 | 0.1737 | 4.48411 | 14.76 |
| 19.8757 | 381.32 | 0.0729 | 4.46343 | 6.35 |
| 20.0711 | 807.25 | 0.1141 | 4.42043 | 13.44 |
| 20.4733 | 1742.06 | 0.1290 | 4.33448 | 29.00 |
| 20.6687 | 1240.64 | 0.1056 | 4.29395 | 20.65 |
| 21.0369 | 0.74 | 0.0518 | 4.21960 | 0.01 |
| 21.1979 | 33.92 | 0.1793 | 4.18793 | 0.56 |
| 21.3540 | 43.74 | 0.1344 | 4.15766 | 0.73 |
| 21.6121 | 305.52 | 0.0968 | 4.10858 | 5.09 |
| 21.8147 | 540.57 | 0.1072 | 4.07089 | 9.00 |
| 21.9791 | 159.88 | 0.1039 | 4.04082 | 2.66 |
| 22.1618 | 513.90 | 0.1051 | 4.00790 | 8.55 |
| 22.3525 | 61.40 | 0.0657 | 3.97413 | 1.02 |
| 22.8539 | 37.05 | 0.0742 | 3.88807 | 0.62 |
| 23.3932 | 200.68 | 0.1227 | 3.79964 | 3.34 |
| 23.6397 | 156.66 | 0.1202 | 3.76058 | 2.61 |
| 23.9268 | 428.67 | 0.1105 | 3.71611 | 7.14 |
| 24.3389 | 328.75 | 0.1155 | 3.65411 | 5.47 |
| 24.7113 | 331.30 | 0.1200 | 3.59988 | 5.51 |
| 24.9550 | 62.15 | 0.0927 | 3.56528 | 1.03 |
| 25.2897 | 211.57 | 0.1291 | 3.51885 | 3.52 |
| 25.4376 | 169.24 | 0.1096 | 3.49871 | 2.82 |
| 25.8122 | 36.88 | 0.0881 | 3.44878 | 0.61 |
| 25.9714 | 15.20 | 0.0800 | 3.42800 | 0.25 |
| 26.2015 | 31.68 | 0.0918 | 3.39842 | 0.53 |
| 26.4196 | 125.76 | 0.1793 | 3.37086 | 2.09 |
| 26.6772 | 116.22 | 0.1360 | 3.33889 | 1.93 |
| 26.8670 | 76.47 | 0.0946 | 3.31573 | 1.27 |
| 27.2060 | 39.25 | 0.0562 | 3.27518 | 0.65 |
| 27.5097 | 18.13 | 0.0725 | 3.23970 | 0.30 |
| 27.5920 | 56.57 | 0.0580 | 3.23023 | 0.94 |
| 27.8344 | 39.48 | 0.0753 | 3.20264 | 0.66 |
| 28.1929 | 33.08 | 0.1112 | 3.16273 | 0.55 |
| 28.5905 | 60.75 | 0.0770 | 3.11965 | 1.01 |
| 28.6859 | 42.86 | 0.1049 | 3.10949 | 0.71 |
| 28.9554 | 226.52 | 0.1038 | 3.08115 | 3.77 |
| 29.1552 | 35.45 | 0.1707 | 3.06050 | 0.59 |
| 29.4814 | 135.08 | 0.0746 | 3.02737 | 2.25 |
| 29.8289 | 39.97 | 0.1139 | 2.99289 | 0.67 |
| 29.9000 | 118.33 | 0.1130 | 2.98593 | 1.97 |
| 30.2913 | 97.02 | 0.0969 | 2.94825 | 1.62 |
| 30.5190 | 54.54 | 0.1374 | 2.92676 | 0.91 |
| 30.8570 | 10.99 | 0.0903 | 2.89548 | 0.18 |
| 31.0553 | 8.22 | 0.0643 | 2.87743 | 0.14 |
| 31.4803 | 35.93 | 0.1386 | 2.83955 | 0.60 |
| 31.7951 | 38.63 | 0.1003 | 2.81215 | 0.64 |
| 31.9735 | 39.58 | 0.1462 | 2.79687 | 0.66 |
| 32.2205 | 17.72 | 0.1060 | 2.77599 | 0.30 |
| 32.6043 | 197.75 | 0.1032 | 2.74418 | 3.29 |
| 32.6992 | 21.33 | 0.1756 | 2.73643 | 0.36 |
| 32.8131 | 20.29 | 0.0368 | 2.72719 | 0.34 |
| 32.9603 | 25.48 | 0.0627 | 2.71535 | 0.42 |
| 33.3855 | 62.25 | 0.0845 | 2.68173 | 1.04 |
| 33.4425 | 47.15 | 0.0695 | 2.67729 | 0.78 |
| 33.6384 | 15.66 | 0.1528 | 2.66214 | 0.26 |
| 33.7877 | 25.88 | 0.0569 | 2.65072 | 0.43 |
| 34.0886 | 30.95 | 0.1460 | 2.62801 | 0.52 |
| 34.1889 | 16.52 | 0.1200 | 2.62053 | 0.27 |
| 34.5339 | 13.81 | 0.0894 | 2.59514 | 0.23 |
| 34.8098 | 4.78 | 0.0458 | 2.57520 | 0.08 |
| 35.2057 | 15.02 | 0.1394 | 2.54714 | 0.25 |
| 35.3966 | 14.69 | 0.0562 | 2.53384 | 0.24 |
| 35.5611 | 13.37 | 0.0715 | 2.52250 | 0.22 |
| 35.8722 | 50.79 | 0.1639 | 2.50133 | 0.85 |
| 36.1408 | 34.62 | 0.0834 | 2.48335 | 0.58 |
| 36.2346 | 14.89 | 0.0639 | 2.47714 | 0.25 |
| 36.6912 | 48.97 | 0.1988 | 2.44735 | 0.82 |
| 36.9067 | 36.55 | 0.0807 | 2.43355 | 0.61 |
| 37.0523 | 29.84 | 0.0913 | 2.42433 | 0.50 |
| 37.2835 | 25.19 | 0.2042 | 2.40982 | 0.42 |
| 37.3509 | 14.59 | 0.0260 | 2.40562 | 0.24 |
| 37.5648 | 21.66 | 0.0346 | 2.39242 | 0.36 |
| 37.7366 | 22.67 | 0.0575 | 2.38192 | 0.38 |
| 38.0512 | 21.87 | 0.1420 | 2.36295 | 0.36 |
| 38.4007 | 14.88 | 0.0560 | 2.34224 | 0.25 |
| 38.6711 | 22.82 | 0.1323 | 2.32649 | 0.38 |
| 38.9600 | 24.73 | 0.2954 | 2.30990 | 0.41 |
| 39.2129 | 26.43 | 0.1017 | 2.29558 | 0.44 |
| 39.9616 | 22.66 | 0.0402 | 2.25428 | 0.38 |

TABLE 9

| Pos. | Height | FWHM Left | d-spacing | Rel. Int. |
|---|---|---|---|---|
| [°2θ] | [cts] | [°2θ] | [Å] | [%] |
| 8.1017 | 3057.89 | 0.1793 | 10.90425 | 100.00 |
| 9.0729 | 183.68 | 0.1838 | 9.73910 | 6.01 |
| 10.5090 | 21.27 | 0.4812 | 8.41123 | 0.70 |
| 11.4588 | 132.65 | 0.1685 | 7.71609 | 4.34 |
| 12.2590 | 248.20 | 0.1883 | 7.21416 | 8.12 |
| 12.6131 | 18.74 | 0.0684 | 7.01241 | 0.61 |
| 14.1731 | 65.12 | 0.3015 | 6.24387 | 2.13 |
| 14.3170 | 249.38 | 0.1863 | 6.18147 | 8.16 |
| 15.9299 | 428.32 | 0.1813 | 5.55902 | 14.01 |
| 16.1494 | 360.97 | 0.1788 | 5.48394 | 11.80 |
| 16.7662 | 201.18 | 0.2081 | 5.28358 | 6.58 |
| 17.4807 | 26.33 | 0.1546 | 5.06919 | 0.86 |
| 17.9701 | 137.97 | 0.1873 | 4.93222 | 4.51 |
| 18.1203 | 124.35 | 0.1711 | 4.89169 | 4.07 |
| 18.8513 | 18.98 | 0.2773 | 4.70362 | 0.62 |
| 19.2757 | 379.24 | 0.1917 | 4.60100 | 12.40 |
| 19.8775 | 51.48 | 0.1351 | 4.46304 | 1.68 |
| 19.9542 | 78.85 | 0.3668 | 4.44606 | 2.58 |
| 20.2819 | 479.24 | 0.2216 | 4.37495 | 15.67 |
| 20.7732 | 91.54 | 0.2293 | 4.27258 | 2.99 |
| 21.2891 | 245.98 | 0.2386 | 4.17018 | 8.04 |
| 21.9031 | 71.35 | 0.3007 | 4.05465 | 2.33 |
| 22.4588 | 17.93 | 0.2884 | 3.95556 | 0.59 |
| 23.0401 | 32.14 | 0.1689 | 3.85707 | 1.05 |
| 23.2667 | 19.91 | 0.1317 | 3.82001 | 0.65 |
| 23.6178 | 266.35 | 0.2219 | 3.76402 | 8.71 |
| 23.8171 | 51.38 | 0.3014 | 3.73296 | 1.68 |
| 24.5567 | 54.04 | 0.3921 | 3.62219 | 1.77 |
| 25.1125 | 35.04 | 0.0971 | 3.54327 | 1.15 |
| 25.5539 | 26.82 | 0.0747 | 3.48305 | 0.88 |
| 25.7689 | 25.26 | 0.1930 | 3.45448 | 0.83 |
| 26.0916 | 33.14 | 0.2213 | 3.41248 | 1.08 |
| 26.2686 | 17.22 | 0.2119 | 3.38989 | 0.56 |
| 26.9759 | 18.05 | 0.2042 | 3.30259 | 0.59 |
| 27.6659 | 29.61 | 0.1088 | 3.22177 | 0.97 |
| 27.9750 | 23.42 | 0.2306 | 3.18687 | 0.77 |
| 28.2452 | 23.14 | 0.0336 | 3.15699 | 0.76 |
| 28.7622 | 14.41 | 0.1915 | 3.10142 | 0.47 |
| 29.0400 | 54.47 | 0.2638 | 3.07237 | 1.78 |
| 29.6738 | 29.31 | 0.3564 | 3.00818 | 0.96 |
| 30.1738 | 7.86 | 0.2616 | 2.95946 | 0.26 |
| 30.3334 | 9.56 | 0.2501 | 2.94425 | 0.31 |
| 31.9219 | 8.74 | 0.2102 | 2.80127 | 0.29 |
| 32.3056 | 15.59 | 0.2412 | 2.76887 | 0.51 |
| 32.5770 | 11.81 | 0.1678 | 2.74642 | 0.39 |
| 32.8648 | 20.31 | 0.0929 | 2.72302 | 0.66 |
| 33.0428 | 40.46 | 0.0854 | 2.70875 | 1.32 |
| 33.1478 | 39.02 | 0.0824 | 2.70042 | 1.28 |
| 33.3658 | 9.49 | 0.0570 | 2.68327 | 0.31 |
| 33.5849 | 19.25 | 0.1372 | 2.66627 | 0.63 |
| 33.7474 | 17.78 | 0.0624 | 2.65380 | 0.58 |
| 34.3982 | 14.58 | 0.1628 | 2.60506 | 0.48 |
| 35.0901 | 9.35 | 0.2198 | 2.55527 | 0.31 |
| 35.1955 | 25.15 | 0.0939 | 2.54786 | 0.82 |
| 35.4448 | 5.57 | 0.0260 | 2.53051 | 0.18 |
| 35.6936 | 0.00 | 0.0260 | 2.51343 | 0.00 |
| 36.2210 | 11.49 | 0.1504 | 2.47803 | 0.38 |
| 36.3862 | 22.80 | 0.1259 | 2.46717 | 0.75 |
| 36.6073 | 6.69 | 0.0381 | 2.45277 | 0.22 |
| 36.7950 | 16.96 | 0.1306 | 2.44069 | 0.55 |
| 36.9399 | 18.76 | 0.1355 | 2.43144 | 0.61 |

TABLE 10

| Pos. | Height | FWHM Left | d-spacing | Rel. Int. |
|---|---|---|---|---|
| [°2θ] | [cts] | [°2θ] | [Å] | [%] |
| 7.1790 | 2260.99 | 0.1838 | 12.30360 | 27.34 |
| 8.8692 | 157.72 | 0.1931 | 9.96229 | 1.91 |
| 10.1741 | 1017.22 | 0.1779 | 8.68730 | 12.30 |
| 10.4436 | 77.65 | 0.0601 | 8.46378 | 0.94 |
| 11.3192 | 385.84 | 0.1948 | 7.81095 | 4.67 |
| 12.4666 | 1354.98 | 0.1951 | 7.09446 | 16.38 |
| 12.7068 | 49.72 | 0.0461 | 6.96089 | 0.60 |
| 13.5997 | 181.39 | 0.1706 | 6.50582 | 2.19 |
| 14.3971 | 4020.63 | 0.1754 | 6.14725 | 48.62 |
| 15.3915 | 1217.34 | 0.1756 | 5.75225 | 14.72 |
| 16.9635 | 482.48 | 0.3310 | 5.22255 | 5.83 |
| 17.2249 | 3937.74 | 0.1713 | 5.14389 | 47.61 |
| 17.5655 | 3289.51 | 0.1912 | 5.04491 | 39.78 |
| 17.9382 | 322.37 | 0.2437 | 4.94094 | 3.90 |
| 18.2919 | 1273.06 | 0.1845 | 4.84618 | 15.39 |
| 18.9703 | 80.99 | 0.1596 | 4.67436 | 0.98 |
| 19.4642 | 213.32 | 0.2630 | 4.55686 | 2.58 |
| 19.6933 | 8270.09 | 0.1884 | 4.50435 | 100.00 |
| 19.9206 | 733.36 | 0.0529 | 4.45348 | 8.87 |
| 20.4205 | 6755.29 | 0.1706 | 4.34557 | 81.68 |
| 20.8788 | 71.19 | 0.2338 | 4.25121 | 0.86 |
| 21.3197 | 530.43 | 0.1476 | 4.16427 | 6.41 |
| 21.6883 | 168.56 | 0.1759 | 4.09433 | 2.04 |
| 22.1527 | 62.89 | 0.2397 | 4.00953 | 0.76 |
| 22.3479 | 88.24 | 0.1482 | 3.97495 | 1.07 |
| 22.7530 | 382.80 | 0.2076 | 3.90509 | 4.63 |
| 23.0711 | 188.68 | 0.1361 | 3.85196 | 2.28 |
| 23.3868 | 794.30 | 0.2034 | 3.80067 | 9.60 |
| 23.6167 | 115.84 | 0.0696 | 3.76419 | 1.40 |
| 23.9593 | 139.80 | 0.2060 | 3.71114 | 1.69 |
| 24.0969 | 438.35 | 0.1650 | 3.69025 | 5.30 |
| 24.6760 | 354.71 | 0.1816 | 3.60494 | 4.29 |
| 25.0910 | 388.86 | 0.1859 | 3.54625 | 4.70 |
| 25.4785 | 56.63 | 0.1992 | 3.49319 | 0.68 |
| 25.6846 | 247.80 | 0.1673 | 3.46563 | 3.00 |
| 26.8707 | 143.81 | 0.2641 | 3.31528 | 1.74 |
| 26.9673 | 599.21 | 0.2109 | 3.30362 | 7.25 |
| 27.2332 | 230.31 | 0.1321 | 3.27197 | 2.78 |
| 27.4238 | 202.04 | 0.1592 | 3.24966 | 2.44 |
| 27.7001 | 98.29 | 0.0889 | 3.21787 | 1.19 |
| 27.8624 | 132.17 | 0.1317 | 3.19949 | 1.60 |
| 27.9677 | 296.43 | 0.1539 | 3.18769 | 3.58 |
| 28.0527 | 80.77 | 0.1060 | 3.17822 | 0.98 |
| 28.3918 | 182.81 | 0.2039 | 3.14103 | 2.21 |
| 28.5242 | 526.91 | 0.1617 | 3.12675 | 6.37 |
| 28.6406 | 205.38 | 0.1106 | 3.11431 | 2.48 |
| 29.0423 | 309.23 | 0.1832 | 3.07213 | 3.74 |
| 29.4163 | 1042.34 | 0.1841 | 3.03392 | 12.60 |
| 29.9947 | 191.78 | 0.1410 | 2.97672 | 2.32 |
| 30.2520 | 100.74 | 0.1548 | 2.95199 | 1.22 |
| 30.5164 | 0.65 | 0.0260 | 2.92701 | 0.01 |
| 30.6987 | 50.85 | 0.1218 | 2.91004 | 0.61 |
| 30.8552 | 742.46 | 0.1756 | 2.89564 | 8.98 |
| 31.0211 | 58.33 | 0.0369 | 2.88053 | 0.71 |
| 31.3351 | 74.85 | 0.1713 | 2.85238 | 0.91 |
| 31.6014 | 188.70 | 0.1542 | 2.82894 | 2.28 |
| 31.7525 | 46.32 | 0.0620 | 2.81583 | 0.56 |
| 32.0726 | 362.41 | 0.2127 | 2.78845 | 4.38 |
| 32.2515 | 87.78 | 0.1577 | 2.77339 | 1.06 |
| 32.5077 | 91.80 | 0.2000 | 2.75212 | 1.11 |
| 33.6377 | 5.76 | 0.0260 | 2.66220 | 0.07 |
| 33.9314 | 20.21 | 0.1405 | 2.63982 | 0.24 |
| 34.2219 | 263.64 | 0.1923 | 2.61808 | 3.19 |
| 34.4222 | 90.34 | 0.0832 | 2.60330 | 1.09 |
| 34.5908 | 39.65 | 0.0681 | 2.59100 | 0.48 |
| 34.7556 | 30.25 | 0.1011 | 2.57909 | 0.37 |
| 34.8715 | 43.78 | 0.1313 | 2.57078 | 0.53 |
| 35.2100 | 172.46 | 0.1633 | 2.54683 | 2.09 |
| 35.7859 | 41.13 | 0.1624 | 2.50716 | 0.50 |
| 36.0107 | 76.07 | 0.2765 | 2.49202 | 0.92 |
| 36.2719 | 88.12 | 0.1082 | 2.47467 | 1.07 |
| 36.5325 | 130.87 | 0.1921 | 2.45762 | 1.58 |
| 36.5910 | 14.46 | 0.3267 | 2.45383 | 0.17 |
| 37.1148 | 200.61 | 0.1652 | 2.42039 | 2.43 |
| 37.2257 | 135.80 | 0.0922 | 2.41343 | 1.64 |
| 37.6389 | 103.34 | 0.1682 | 2.38788 | 1.25 |
| 37.7824 | 116.75 | 0.0768 | 2.37914 | 1.41 |
| 37.8790 | 144.05 | 0.1494 | 2.37329 | 1.74 |
| 38.0279 | 291.52 | 0.1671 | 2.36434 | 3.53 |
| 38.5602 | 102.32 | 0.1250 | 2.33292 | 1.24 |
| 38.6468 | 120.94 | 0.2089 | 2.32789 | 1.46 |
| 38.7940 | 32.19 | 0.0686 | 2.31940 | 0.39 |
| 38.9252 | 56.36 | 0.0954 | 2.31188 | 0.68 |
| 39.1428 | 47.52 | 0.1741 | 2.29953 | 0.57 |
| 39.6636 | 40.04 | 0.3077 | 2.27053 | 0.48 |
| 39.7687 | 72.00 | 0.1933 | 2.26477 | 0.87 |
| 39.9750 | 73.97 | 0.1885 | 2.25355 | 0.89 |

TABLE 10A

| Pos. | Height | FWHM Left | d-spacing | Rel. Int. |
|---|---|---|---|---|
| [°2θ] | [cts] | [°2θ] | [Å] | [%] |
| 2.0469 | 42.55 | 0.5856 | 43.12501 | 47.99 |
| 5.4559 | 88.67 | 0.1540 | 16.18482 | 100.00 |
| 7.3811 | 7.17 | 0.1468 | 11.96717 | 8.09 |
| 8.4574 | 61.93 | 0.1722 | 10.44644 | 69.85 |
| 9.5091 | 21.22 | 0.1718 | 9.29335 | 23.93 |
| 9.9161 | 11.02 | 0.0270 | 8.91278 | 12.43 |
| 10.2795 | 21.03 | 0.1844 | 8.59851 | 23.71 |
| 10.8805 | 5.52 | 0.2347 | 8.12484 | 6.23 |
| 11.3202 | 17.19 | 0.1971 | 7.81023 | 19.39 |
| 11.9777 | 31.65 | 0.1742 | 7.38293 | 35.69 |
| 14.2223 | 6.34 | 0.1580 | 6.22238 | 7.15 |
| 14.7269 | 18.31 | 0.1443 | 6.01033 | 20.66 |
| 15.0592 | 9.81 | 0.6895 | 5.87842 | 11.06 |
| 16.1923 | 86.26 | 0.1651 | 5.46954 | 97.29 |
| 16.8005 | 14.05 | 0.1525 | 5.27286 | 15.84 |
| 17.0909 | 31.80 | 0.2409 | 5.18392 | 35.86 |
| 17.7701 | 24.23 | 0.3704 | 4.98727 | 27.33 |
| 18.1531 | 7.91 | 0.1100 | 4.88291 | 8.92 |
| 18.4144 | 35.71 | 0.2549 | 4.81422 | 40.28 |
| 18.7351 | 17.97 | 0.1633 | 4.73253 | 20.27 |
| 19.0193 | 38.75 | 0.2506 | 4.66244 | 43.71 |
| 19.3029 | 3.60 | 0.1990 | 4.59457 | 4.06 |
| 19.6745 | 8.93 | 0.0218 | 4.50862 | 10.07 |
| 20.0450 | 13.24 | 0.4234 | 4.42611 | 14.93 |
| 21.0670 | 62.20 | 0.1876 | 4.21366 | 70.15 |
| 21.7100 | 10.41 | 0.1647 | 4.09029 | 11.75 |
| 23.0699 | 9.19 | 0.3452 | 3.85215 | 10.36 |
| 24.4990 | 7.22 | 0.2194 | 3.63060 | 8.15 |
| 25.5882 | 4.64 | 0.2173 | 3.47847 | 5.23 |
| 28.7557 | 8.07 | 0.1881 | 3.10210 | 9.10 |

TABLE 11

| Pos. | Height | FWHM Left | d-spacing | Rel. Int. |
|---|---|---|---|---|
| [°2θ] | [cts] | [°2θ] | [Å] | [%] |
| 7.4531 | 21.81 | 0.1323 | 11.85174 | 2.23 |
| 8.2814 | 4.43 | 0.1467 | 10.66810 | 0.45 |
| 10.1478 | 6.82 | 0.1159 | 8.70982 | 0.70 |
| 12.4966 | 49.62 | 0.1147 | 7.07751 | 5.08 |
| 13.0012 | 169.49 | 0.1176 | 6.80394 | 17.35 |
| 14.2626 | 123.59 | 0.1081 | 6.20493 | 12.65 |
| 14.8453 | 977.07 | 0.1152 | 5.96264 | 100.00 |
| 15.6931 | 35.54 | 0.1249 | 5.64238 | 3.64 |
| 16.5206 | 187.82 | 0.1115 | 5.36158 | 19.22 |
| 16.9336 | 54.70 | 0.1047 | 5.23173 | 5.60 |
| 17.9431 | 199.52 | 0.0978 | 4.93958 | 20.42 |
| 18.2082 | 186.58 | 0.0994 | 4.86827 | 19.10 |
| 18.6614 | 43.47 | 0.0986 | 4.75105 | 4.45 |
| 19.2613 | 142.78 | 0.1152 | 4.60441 | 14.61 |
| 19.3561 | 55.59 | 0.1099 | 4.58206 | 5.69 |
| 19.6949 | 737.51 | 0.1034 | 4.50400 | 75.48 |
| 20.0893 | 87.64 | 0.1078 | 4.41647 | 8.97 |
| 20.2978 | 5.00 | 0.0873 | 4.37156 | 0.51 |
| 20.8064 | 10.08 | 0.0232 | 4.26583 | 1.03 |
| 21.4332 | 85.73 | 0.1061 | 4.14249 | 8.77 |
| 21.7370 | 43.01 | 0.1009 | 4.08526 | 4.40 |
| 22.2697 | 17.26 | 0.0455 | 3.98874 | 1.77 |
| 22.4400 | 9.43 | 0.2670 | 3.95885 | 0.97 |
| 22.6465 | 25.85 | 0.1126 | 3.92321 | 2.65 |
| 23.4520 | 24.26 | 0.1099 | 3.79025 | 2.48 |
| 24.1020 | 41.18 | 0.0810 | 3.68948 | 4.21 |
| 24.5020 | 103.41 | 0.0995 | 3.63016 | 10.58 |
| 24.6022 | 75.04 | 0.0982 | 3.61559 | 7.68 |
| 24.6653 | 38.58 | 0.0200 | 3.60649 | 3.95 |
| 24.8664 | 4.16 | 0.0445 | 3.57778 | 0.43 |
| 25.0456 | 17.98 | 0.0554 | 3.55258 | 1.84 |
| 25.0989 | 3.23 | 0.3048 | 3.54515 | 0.33 |
| 25.5569 | 79.50 | 0.0789 | 3.48266 | 8.14 |
| 25.6094 | 53.11 | 0.0543 | 3.47563 | 5.44 |
| 26.1062 | 39.01 | 0.1493 | 3.41061 | 3.99 |
| 26.1593 | 15.47 | 0.0626 | 3.40380 | 1.58 |
| 26.3581 | 56.02 | 0.1112 | 3.37858 | 5.73 |
| 26.7303 | 64.96 | 0.0997 | 3.33238 | 6.65 |
| 26.7938 | 24.68 | 0.1349 | 3.32462 | 2.53 |
| 26.8594 | 32.87 | 0.0476 | 3.31665 | 3.36 |
| 27.1328 | 14.26 | 0.1052 | 3.28385 | 1.46 |
| 27.2137 | 15.69 | 0.0531 | 3.27427 | 1.61 |
| 27.4122 | 10.35 | 0.0200 | 3.25101 | 1.06 |
| 27.9317 | 7.36 | 0.0294 | 3.19171 | 0.75 |
| 28.1478 | 1.07 | 0.0266 | 3.16769 | 0.11 |
| 28.5938 | 28.71 | 0.0750 | 3.11930 | 2.94 |
| 28.6767 | 12.35 | 0.0508 | 3.11046 | 1.26 |
| 29.0132 | 10.30 | 0.1381 | 3.07515 | 1.05 |
| 29.5914 | 4.53 | 0.0200 | 3.01636 | 0.46 |
| 29.8294 | 22.35 | 0.0699 | 2.99284 | 2.29 |
| 30.2256 | 12.79 | 0.1429 | 2.95450 | 1.31 |
| 30.5527 | 7.54 | 0.0254 | 2.92361 | 0.77 |
| 31.2791 | 8.22 | 0.1670 | 2.85736 | 0.84 |
| 31.4053 | 9.50 | 0.0753 | 2.84616 | 0.97 |
| 31.5853 | 14.47 | 0.0884 | 2.83035 | 1.48 |
| 31.6876 | 10.50 | 0.0559 | 2.82144 | 1.07 |
| 31.7654 | 13.64 | 0.1543 | 2.81471 | 1.40 |
| 32.0089 | 57.09 | 0.1033 | 2.79385 | 5.84 |
| 32.1875 | 23.31 | 0.1251 | 2.77876 | 2.39 |
| 32.3469 | 23.93 | 0.0781 | 2.76542 | 2.45 |
| 32.6974 | 6.02 | 0.1300 | 2.73658 | 0.62 |
| 33.2643 | 8.34 | 0.1016 | 2.69122 | 0.85 |
| 33.6289 | 13.11 | 0.1134 | 2.66288 | 1.34 |
| 33.9600 | 4.95 | 0.1392 | 2.63767 | 0.51 |
| 34.4445 | 6.73 | 0.0589 | 2.60167 | 0.69 |
| 34.8418 | 8.47 | 0.1734 | 2.57290 | 0.87 |
| 35.5276 | 7.57 | 0.0837 | 2.52479 | 0.78 |
| 35.6785 | 12.28 | 0.0631 | 2.51446 | 1.26 |
| 36.0295 | 13.28 | 0.0754 | 2.49077 | 1.36 |
| 36.2285 | 20.69 | 0.0956 | 2.47754 | 2.12 |
| 36.3984 | 9.83 | 0.0852 | 2.46636 | 1.01 |
| 36.6959 | 12.73 | 0.1364 | 2.44705 | 1.30 |
| 36.8541 | 6.12 | 0.0664 | 2.43690 | 0.63 |
| 37.0103 | 5.42 | 0.0860 | 2.42698 | 0.56 |
| 37.1242 | 10.82 | 0.0978 | 2.41980 | 1.11 |
| 37.5239 | 48.65 | 0.0694 | 2.39493 | 4.98 |
| 37.8768 | 8.45 | 0.1294 | 2.37343 | 0.86 |
| 38.1996 | 14.33 | 0.1104 | 2.35411 | 1.47 |
| 38.4463 | 13.45 | 0.1804 | 2.33957 | 1.38 |
| 38.7729 | 10.08 | 0.0908 | 2.32061 | 1.03 |
| 39.0315 | 7.95 | 0.2263 | 2.30583 | 0.81 |
| 39.4762 | 14.53 | 0.0729 | 2.28087 | 1.49 |
| 39.5198 | 5.64 | 0.1481 | 2.27846 | 0.58 |
| 39.8794 | 21.14 | 0.1286 | 2.25873 | 2.16 |

TABLE 12

| Pos. | Height | FWHM Left | d-spacing | Rel. Int. |
|---|---|---|---|---|
| [°2θ] | [cts] | [°2θ] | [Å] | [%] |
| 5.9249 | 202.32 | 0.1813 | 14.90465 | 100.00 |
| 7.1565 | 19.18 | 0.2175 | 12.34224 | 9.48 |
| 7.4641 | 6.22 | 0.2181 | 11.83423 | 3.08 |
| 10.7780 | 10.13 | 0.2324 | 8.20186 | 5.01 |
| 11.3625 | 7.44 | 0.2264 | 7.78126 | 3.68 |
| 11.7710 | 150.16 | 0.2262 | 7.51214 | 74.22 |
| 13.3163 | 28.42 | 0.1921 | 6.64366 | 14.05 |
| 14.2336 | 52.64 | 0.2476 | 6.21747 | 26.02 |
| 14.8665 | 72.07 | 0.2098 | 5.95419 | 35.62 |
| 15.7478 | 4.24 | 0.3113 | 5.62290 | 2.10 |
| 16.6846 | 43.92 | 0.2357 | 5.30925 | 21.71 |
| 16.9223 | 20.92 | 0.2093 | 5.23520 | 10.34 |
| 17.1997 | 46.19 | 0.2910 | 5.15138 | 22.83 |
| 17.6136 | 16.42 | 0.1820 | 5.03124 | 8.12 |
| 18.2544 | 27.37 | 0.2025 | 4.85605 | 13.53 |
| 19.4867 | 33.31 | 0.2093 | 4.55165 | 16.46 |
| 19.7449 | 10.40 | 0.1108 | 4.49270 | 5.14 |
| 19.9329 | 22.66 | 0.2701 | 4.45075 | 11.20 |
| 20.1557 | 15.24 | 0.6129 | 4.40206 | 7.53 |
| 20.6330 | 147.17 | 0.2139 | 4.30129 | 72.74 |
| 21.1675 | 37.46 | 0.3070 | 4.19386 | 18.51 |
| 21.3207 | 15.88 | 0.3730 | 4.16409 | 7.85 |
| 21.9791 | 100.55 | 0.2175 | 4.04080 | 49.70 |
| 23.2303 | 11.54 | 0.1970 | 3.82592 | 5.70 |
| 23.5375 | 3.26 | 0.1945 | 3.77668 | 1.61 |
| 23.8678 | 5.73 | 0.0481 | 3.72516 | 2.83 |
| 24.1114 | 35.97 | 0.1993 | 3.68807 | 17.78 |
| 24.2613 | 13.88 | 0.1038 | 3.66562 | 6.86 |
| 24.7266 | 5.67 | 0.1873 | 3.59769 | 2.80 |
| 24.7676 | 12.06 | 0.1807 | 3.59182 | 5.96 |
| 25.4906 | 15.48 | 0.2501 | 3.49156 | 7.65 |
| 25.7644 | 26.19 | 0.2551 | 3.45507 | 12.94 |
| 26.6405 | 5.60 | 0.4034 | 3.34341 | 2.77 |
| 27.0950 | 2.86 | 0.0425 | 3.28835 | 1.41 |
| 27.6047 | 10.04 | 0.0200 | 3.22878 | 4.96 |
| 27.7105 | 15.48 | 0.1518 | 3.21669 | 7.65 |
| 27.8890 | 6.50 | 0.1285 | 3.19650 | 3.21 |
| 28.2754 | 5.53 | 0.2107 | 3.15369 | 2.73 |
| 28.6297 | 5.62 | 0.1932 | 3.11547 | 2.78 |
| 28.8792 | 0.00 | 0.0200 | 3.08911 | 0.00 |
| 29.5138 | 19.19 | 0.2134 | 3.02412 | 9.49 |
| 29.7244 | 9.95 | 0.1369 | 3.00317 | 4.92 |
| 29.8906 | 10.34 | 0.0896 | 2.98685 | 5.11 |
| 30.0808 | 10.07 | 0.0542 | 2.96840 | 4.98 |
| 30.8782 | 2.00 | 0.3802 | 2.89353 | 0.99 |
| 31.1526 | 3.39 | 0.0200 | 2.86867 | 1.68 |
| 31.8860 | 4.91 | 0.2399 | 2.80434 | 2.42 |
| 32.8460 | 8.79 | 0.0763 | 2.72454 | 4.35 |
| 32.9769 | 6.11 | 0.0798 | 2.71402 | 3.02 |
| 33.5242 | 8.31 | 0.2590 | 2.67096 | 4.11 |
| 34.1489 | 3.75 | 0.0615 | 2.62351 | 1.85 |
| 34.2813 | 5.86 | 0.0900 | 2.61368 | 2.90 |
| 34.4181 | 6.88 | 0.0514 | 2.60360 | 3.40 |
| 34.5888 | 4.81 | 0.1900 | 2.59114 | 2.38 |
| 34.7630 | 4.50 | 0.0200 | 2.57856 | 2.23 |
| 34.8640 | 1.99 | 0.1044 | 2.57132 | 0.98 |
| 35.6852 | 5.02 | 0.0258 | 2.51401 | 2.48 |
| 35.8133 | 7.13 | 0.0906 | 2.50530 | 3.53 |
| 35.9531 | 8.53 | 0.0601 | 2.49588 | 4.21 |
| 36.1039 | 5.59 | 0.0200 | 2.48580 | 2.76 |
| 36.1745 | 1.69 | 0.1768 | 2.48111 | 0.83 |
| 36.2156 | 0.65 | 0.0200 | 2.47839 | 0.32 |
| 36.6697 | 1.31 | 0.0200 | 2.44874 | 0.65 |
| 36.7061 | 1.74 | 0.0200 | 2.44639 | 0.86 |
| 36.9709 | 3.06 | 0.0262 | 2.42948 | 1.51 |
| 37.0174 | 3.03 | 0.0247 | 2.42653 | 1.50 |
| 37.3550 | 6.56 | 0.0879 | 2.40538 | 3.24 |
| 39.3306 | 5.29 | 0.0883 | 2.28898 | 2.62 |
| 39.6897 | 5.28 | 0.4011 | 2.26909 | 2.61 |

TABLE 12A

| Pos [°2θ] | d-spacing [Å] | Rel. Int. [%] |
|---|---|---|
| 8.30 | 10.65 | 100 |
| 15.09 | 5.87 | 7 |
| 15.59 | 5.68 | 18 |
| 16.53 | 5.36 | 5 |
| 17.36 | 5.10 | 10 |
| 17.86 | 4.96 | 13 |
| 19.94 | 4.45 | 5 |
| 20.06 | 4.42 | 6 |
| 23.44 | 3.79 | 20 |

### Polarized Light Microscopy (PLM)

The PLM image for crystalline form B isolated from ethyl acetate is shown in FIG. 1, which shows a material having block-like morphology under plane-polarized light. The PLM images for crystalline form C isolated from acetone/water are shown in FIGS. 5A and 5B, which show a material having needle/plate-like morphology under plane polarized light. The PLM image for crystalline form F isolated from aqueous ethanol is shown in FIG. 10, which shows a material having cube-like morphology under plane-polarized light. The PLM image for crystalline hydrogen sulfate form A is shown in FIG. 20, which shows a material having square-like morphology under plane-polarized light. The PLM images for crystalline hydrogen sulfate form B are shown in FIGS. 25A and 25B, which show a material having needle/thin plate-like morphology under plane-polarized light. The PLM image for crystalline sulfate form A is shown in FIG. 58.

### Example 4. Water Solubility of Free Form Solid States at Room Temperature

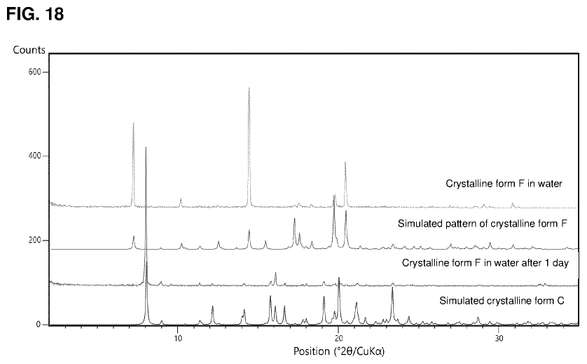

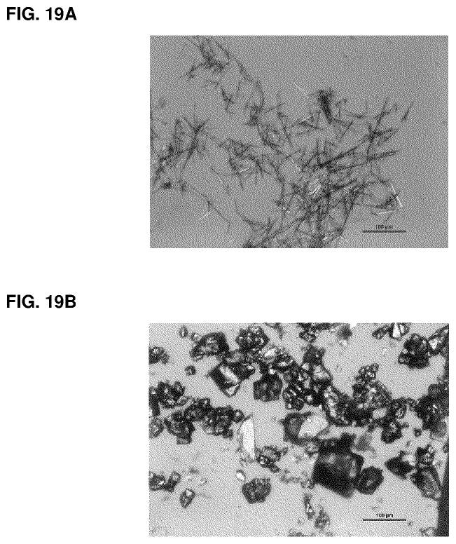

An appropriate amount of crystalline form B and crystalline form F were each weighed into 4 mL vials, and water was added to the solids with increments of 0.1 mL until 0.7 mL was added to each vial. The samples were shaken at 25° C., 200 rpm. XRPD (taken after the crystalline forms were mixed with water, and after 1 day suspended in water) and PLM (after 5 days) were recorded. Crystalline form B converted to crystalline form C in water as confirmed by PLM (FIGS. 19A and 19B) and XRPD (FIG. 18). The water solubility of crystalline form C was about 0.05 mg/mL. Crystalline form F remained unchanged in water, and the solubility was about 0.046 mg/mL. Table 13 summarizes the water solubility of crystalline forms C and F.

TABLE 13

| Crystalline Form | Solubility | XRPD Pattern |
|---|---|---|
| (starting material) | (mg/ml) | (solubility measured on) |
| Crystalline form B | 0.050 | Crystalline form C |
| Crystalline form F | 0.046 | Crystalline form F |

### Example 5. Stability of a Metastable Hydrogen Sulfate Form

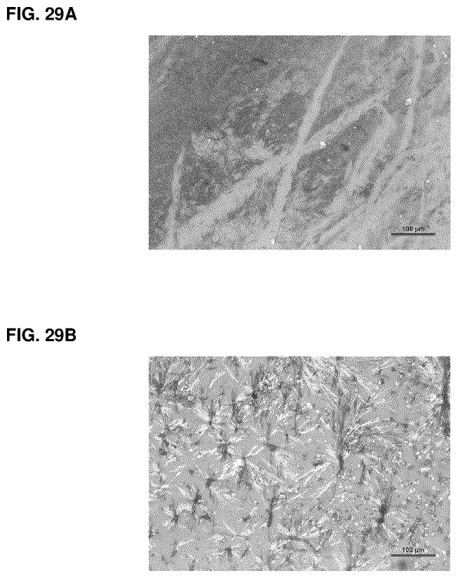

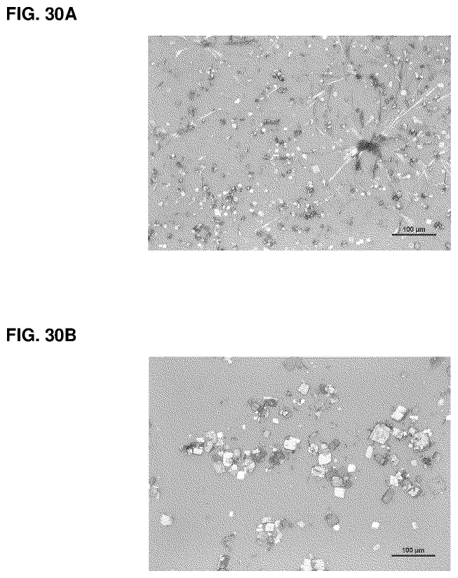

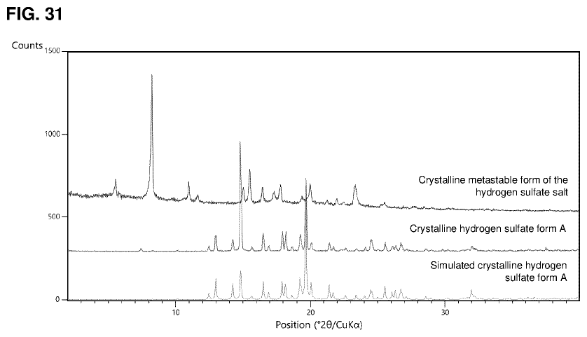

During preparation of crystalline hydrogen sulfate form A in EtOH, the suspension was monitored by PLM (FIGS. 29A, 29B, 30A, and 30B). A metastable form was discovered. The crystalline particles had a fine needle-like morphology, and a different XRPD pattern compared to crystalline hydrogen sulfate form A (FIG. 31). However, after 18 h of stirring the particles converted to square-like crystals. The XRPD recorded on the solids was compared to the pattern simulated by SCXRD, and confirmed crystalline hydrogen sulfate form A was obtained (FIG. 31). No further characterization was conducted to identify this metastable form. Conversion of this form to crystalline hydrogen sulfate form A was accelerated with excess acid.

### Example 6. Form Stability of Crystalline Form F in 0.5% Methylcellulose (v/v) (400 cps)/0.02% Sodium Lauryl Sulfate (v/v)

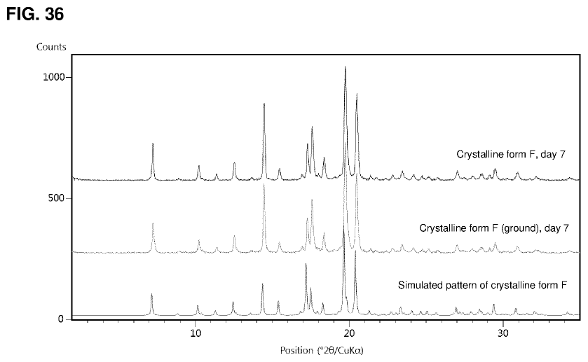

Crystalline form F (56 mg) was weighed in a 20 mL vial and 0.5% methylcellulose (v/v) (400 cps)/0.02% sodium lauryl sulfate (v/v) (14 mL) was added to the solid and stirred at room temperature (500 rpm). Another vial was prepared with the same experimental conditions but with ground crystalline form F instead. The samples were evaluated at designated time points and PLM images were recorded up to 7 days. No form change was obtained after 7 days, as also confirmed by XRPD (FIG. 36). Crystalline form F had very low aqueous solubility, while the other crystalline forms had better aqueous solubility and converted to crystalline form F in the formulations tested.

### Example 7. Form Stability of Crystalline Hydrogen Sulfate Forms A and B in Buffered 0.5% Methylcellulose (v/v) (400 cps)/0.02% Sodium Lauryl Sulfate (v/v)

Crystalline hydrogen sulfate form A was used for form stability studies in 0.5% methylcellulose (v/v) (400 cps)/0.02% sodium lauryl sulfate (v/v) with citrate buffer (pH 4.5) with a concentration of 0.1 mg/mL, 0.2 mg/mL, 0.5 mg/mL and 1 mg/mL with and without stirring. HPLC purity was obtained for the solutions at 3 and 7 days. The purity of the solutions remained in the range 99.8-99.9% (Table 14).

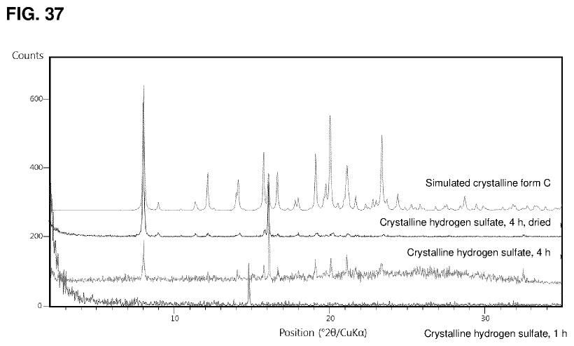

An additional sample of crystalline hydrogen sulfate form A in the same buffered formulation was prepared with a concentration of 1 mg/ml and stirred at 500 rpm at room temperature. The sample was monitored every hour for form assessment. The solution was nearly clear at the first 3 hours. Precipitation of the free form, crystalline form C was observed after 3 hours. XRPD analysis of the precipitate confirmed crystalline form C had formed (FIG. 37).

TABLE 14
0.5% methylcellulose (v/v) (400 cps)/0.02% sodium
lauryl sulfate (v/v) with 50 mM citrate buffer

| | 0.1 mg/mL | | 0.2 mg/mL | | 0.5 mg/mL | | 1 mg/mL | |
|---|---|---|---|---|---|---|---|---|
| | A | | B | | C | | D | |
| Conditions | stir | w/o stir | stir | w/o stir | stir | w/o stir | stir | w/o stir |
| Day 1 | n/a | n/a | n/a | n/a | n/a | n/a | n/a | n/a |
| Day 2 | n/a | n/a | n/a | n/a | n/a | n/a | n/a | n/a |
| Day 3 | 99.82% | 99.82% | 99.89% | 99.89% | 99.91% | 99.91% | 99.93% | 99.89% |
| Day 7 | 99.93% | n/a | 99.86% | n/a | 99.87% | n/a | 99.94% | n/a |

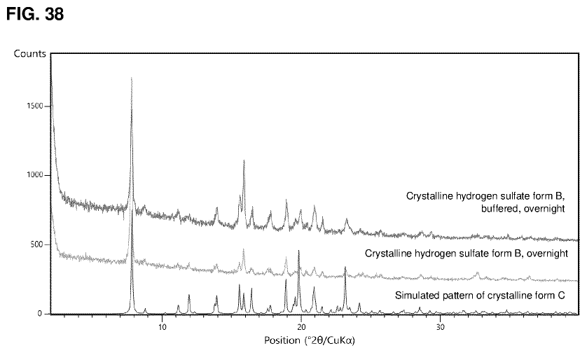

Crystalline hydrogen sulfate form B (10 mg) was suspended in 10 mL of 0.5% methylcellulose (v/v) (400 cps)/0.02% sodium lauryl sulfate (v/v) with (pH 4.5) and without sodium citrate buffer, and stirred at 500 rpm, room temperature, overnight.
The resulting solids were determined to be the free form, crystalline form C as confirmed by XRPD (FIG. 38).

### Example 8. Storage Stability of the Crystalline Forms

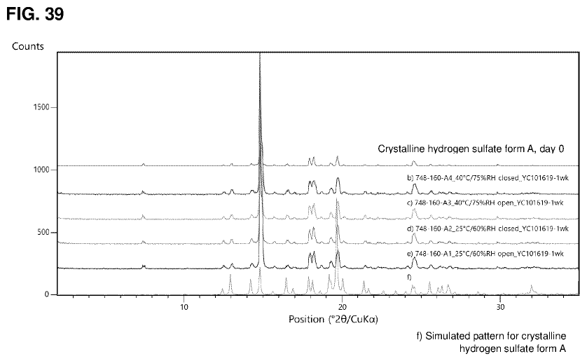

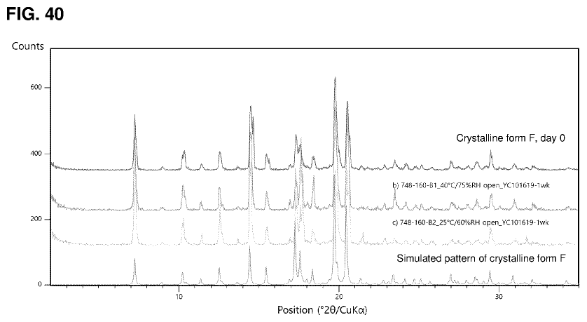

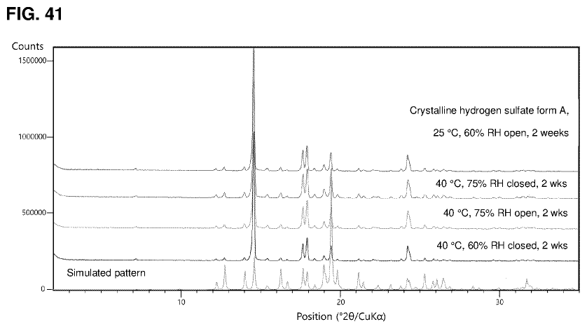

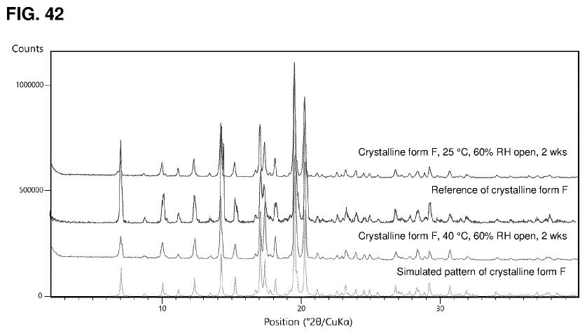

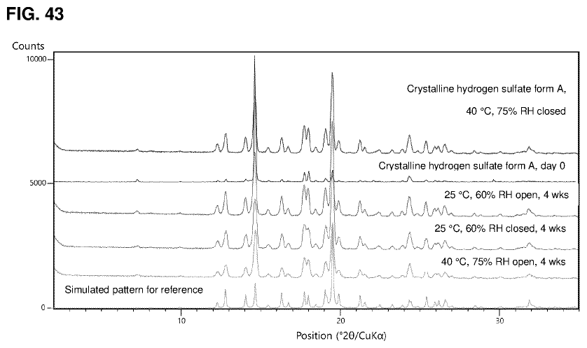

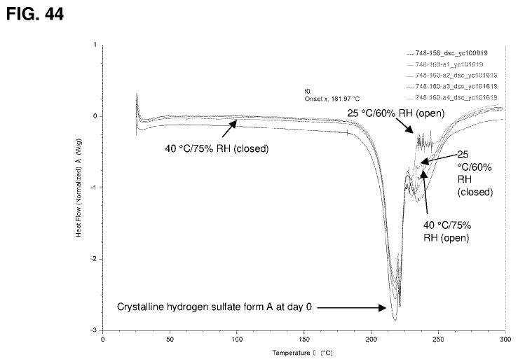

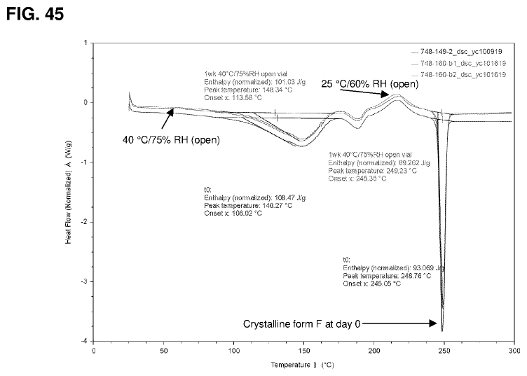

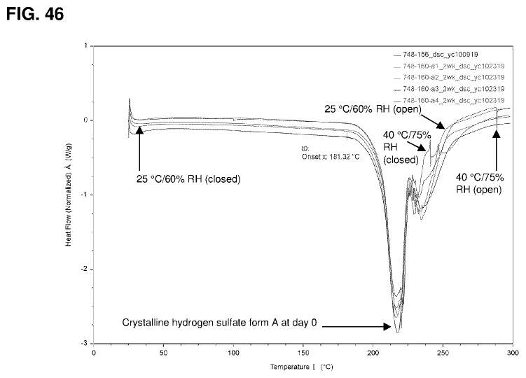

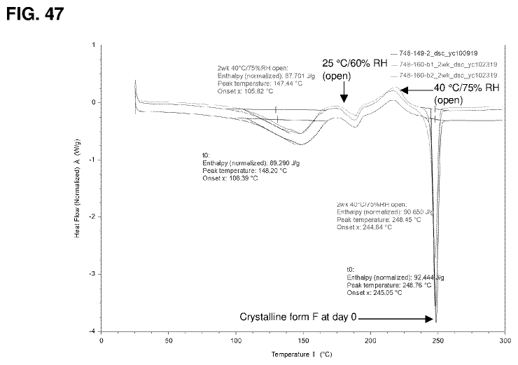

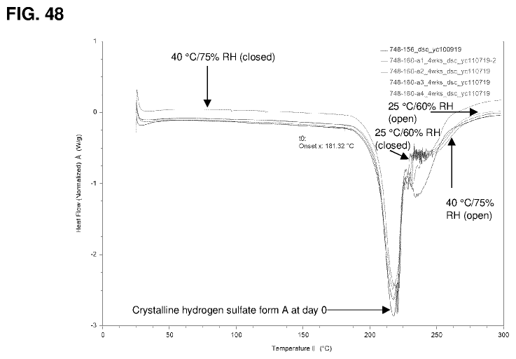

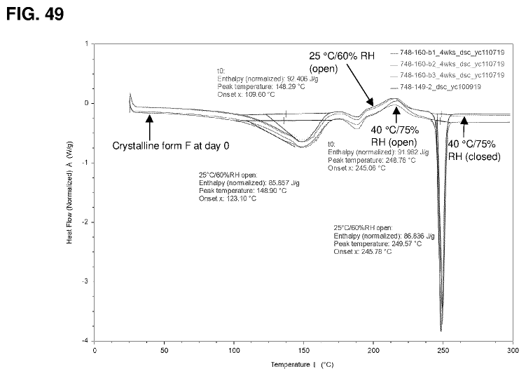

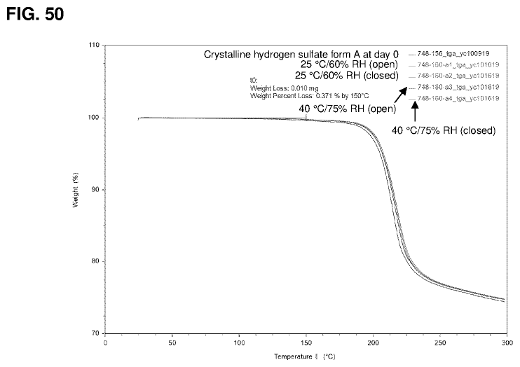

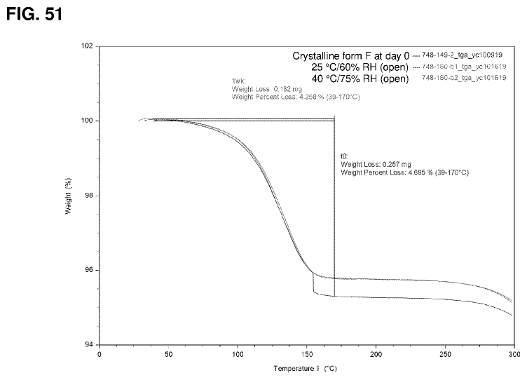

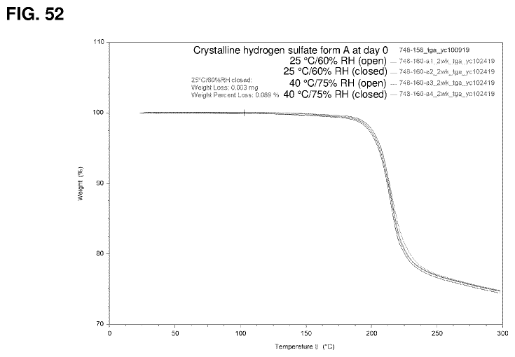

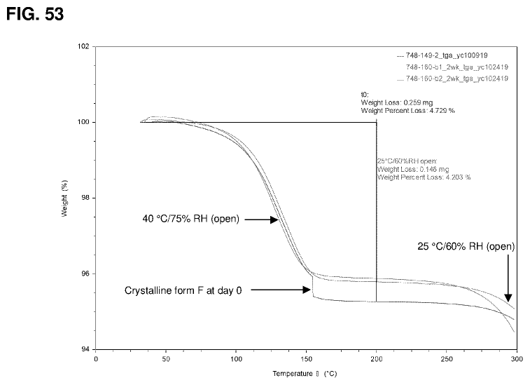

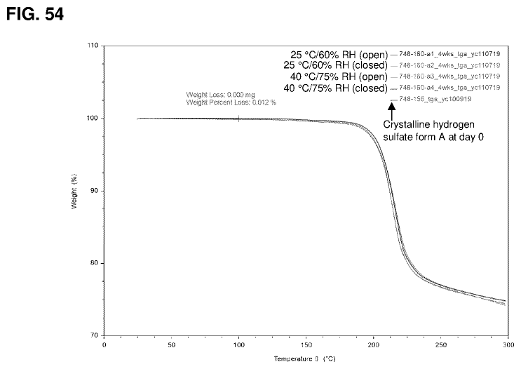

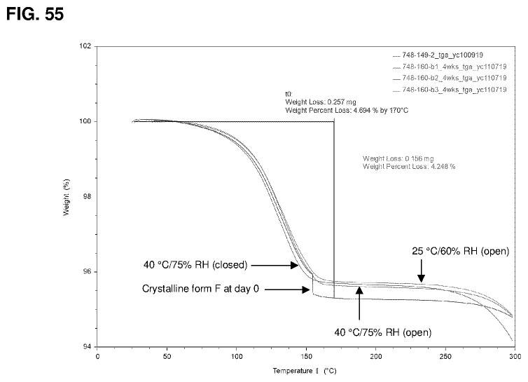

Crystalline Form F and Crystalline Hydrogen Sulfate Form A. 7-day, 14-day, and 4-week stability assessments of crystalline form F and crystalline hydrogen sulfate form A were performed as follows: samples of each form were weighed (about 40 mg) into separate vials and placed under 25° C./60% RH and 400C/75% RH. The forms were evaluated with a closed vial and an open vial. The XRPD of the samples after 1, 2 and 4 weeks of exposure matched with the starting materials (t=0) patterns (FIGS. 39-43). Both crystalline hydrogen sulfate form A and crystalline form F showed similar thermal behavior under DSC compared to t=0 (FIGS. 44-49). TG (FIGS. 50-55) and HPLC data are summarized in Tables 15 (crystalline form F summary) and 16 (crystalline hydrogen sulfate form A).

TABLE 15

| Storage | | | t = 7 d | t = 14 d | t = 4 weeks | |
|---|---|---|---|---|---|---|
| Condition | Analysis | t = 0 | Open vial | Open vial | Open vial | Closed vial |
| 25° C./60% RH | XRPD | Crystalline form | No change | No change | No change | n/a |
| | | F | | | | |
| | TG | 4.7 wt % loss by | 4.2 wt % loss | 4.2 wt % loss | 4.2 wt % loss | n/a |
| | | 170° C. | by 170° C. | by 170° C. | by 170° C. | |
| | DSC | 105° C. (onset | No change | No change | No change | n/a |
| | | (dehydration)) | | | | |
| | HPLC | 99.903% | 99.925% | 100% | 99.94% | n/a |
| 40° C./75% RH | XRPD | Crystalline form | No change | No change | No change | No change |
| | | F | | | | |
| | TG | 4.7 wt % loss by | 4.2 wt % loss | 4.2 wt % loss | 4.2 wt % loss | 4.2 wt % loss |
| | | 170° C. | by 170° C. | by 170° C. | by 170° C. | by 170° C. |
| | DSC | 105° C. (onset | No change | No change | No change | No change |
| | | ((dehydration)) | | | | |
| | HPLC | 99.903% | 99.922% | 100% | 99.94% | 99.94% |

TABLE 16

| | | | t = 7 d | | t = 14 d | | t = 4 weeks | |
|---|---|---|---|---|---|---|---|---|
| Storage | | | | Closed | Open | Closed | Open | Closed |
| Condition | Analysis | t = 0 | Open vial | vial | vial | vial | vial | vial |
| 25° C./60% RH | XRPD | neat | No | No | No | No | No | No |
| | | | change | change | change | change | change | change |
| | TG | Negligible | No | No | No | No | No | No |
| | | (less than | change | change | change | change | change | change |
| | | 0.1% by | | | | | | |
| | | 100° C.) | | | | | | |
| | DSC | 181.9° C. | No | No | No | No | No | No |
| | | (onset) | change | change | change | change | change | change |
| | HPLC | 99.902% | 99.937% | 99.936% | 99.944% | 100% | 99.91% | 99.92% |
| 40° C./75% RH | XRPD | neat | No | No | No | No | No | No |
| | | | change | change | change | change | change | change |
| | TG | Negligible | No | No | No | No | No | No |
| | | (less than | change | change | change | change | change | change |
| | | 0.1% by | | | | | | |
| | | 100° C.) | | | | | | |
| | DSC | 181.9° C. | No | No | No | No | No | No |
| | | (onset) | change | change | change | change | change | change |
| | HPLC | 99.902% | 99.938% | 99.938% | 100% | 100% | 99.91% | 99.92% |

Crystalline Hydrogen Sulfate Form B. The solid-form stability of crystalline hydrogen sulfate form B was tested after one-week exposure to 75% RH at 40° C. Approximately 30 mg of crystalline hydrogen sulfate form B was weighed into a 4 mL vial and covered with a KIMWIPE. The vial was placed inside a 20 mL scintillation vial containing a saturated sodium chloride (NaCl) solution producing 75% RH at 40° C. The vial was sealed with PARAFILM and placed on a hotplate and left undisturbed for one week. After one week, no significant changes were observed (confirmed through XRPD analysis) and the sample remained a flowable, white powder.

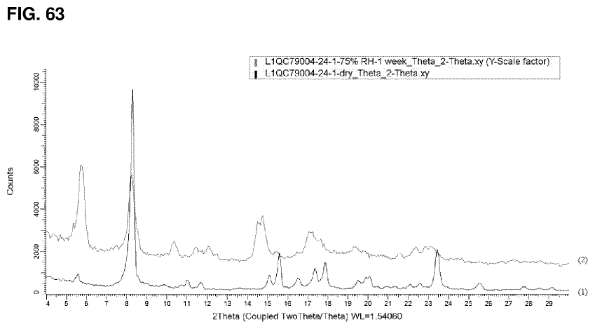

Crystalline Sulfate Form A. The solid-form stability of crystalline sulfate form A was tested after one-week exposure to 75% RH at 40° C. Approximately 30 mg of crystalline sulfate form A was weighed into a 4 mL vial and covered with a KIMWIPE. The vial was placed inside a 20 mL scintillation vial containing a saturated sodium chloride (NaCl) solution producing 75% RH at 40° C. The vial was sealed with PARAFILM and placed on a hotplate and left undisturbed for one week. After one week, no significant changes were observed and the sample remained a flowable, white powder. XRPD analysis showed that crystalline sulfate form A changed to a mixture of crystalline sulfate form A+another crystalline form of a sulfate salt of the compound of formula (I) after one week at 75% RH (FIG. 63).

### Example 9. Free Base Solid State Screening

Solid state screening of the compound of formula (I) was conducted by slurry crystallization. The solubility of crystalline form A in 25 single solvents was measured at room temperature (Table 17). About 20 mg of the starting material was weighed into 2 mL vials, and solvents were added in increments of 0.2 mL. Crystalline form A had a low solubility in the solvents tested except for DMF, DMSO, NMP and DMAc (>100 mg/mL). XRPD data were collected on the resulting solids after 7 days.

TABLE 17

| | Solubility (S) at RT | |
|---|---|---|
| Solvents | (mg/mL) | New Solid State? |
| MeOH | 25 < S < 33.3 | n/a |
| EtOH | 12.4 < S < 25 | n/a |
| IPA | S < 13.3 (thin slurry) | n/a |
| iPAc | S < 2.9 | Crystalline form B |
| EtOAc | S < 16.7 | Crystalline form B |
| nPA | S < 16.2 (thin slurry) | n/a |
| MeCN | S < 16.7 | Crystalline form B |
| MEK | S < 16.7 | Crystalline form B |
| MIBK | S < 16.7 | Crystalline form B |
| MTBE | S < 16.7 | Crystalline form B |
| THF | S < 34.2 | Crystalline form D |
| 1,4-Dioxane | 20 < S < 25 | n/a |
| 2-MeTHF | S < 16.4 | Crystalline form E |
| Heptane | S < 16.7 | Crystalline form A |
| Cyclohexane | S < 16.2 | Crystalline form A |
| Toluene | S < 16.7 | Crystalline form B |
| Water | S < 16.7 | Crystalline form F |
| Acetone | S < 17.1 | Crystalline form B |
| DCM | S < 16.7 | Crystalline form G |
| CPME | S < 16.4 | Crystalline form B |
| DIPE | S < 16.8 | Crystalline form B |
| DMF | S > 100 | n/a |
| DMSO | S > 100 | n/a |
| NMP | S > 100 | n/a |
| DMAc | S > 100 | n/a |

### Example 10. Salt Screening

Salt screening of the compound of formula (I) was conducted with multiple solvents (Table 18). The compound of formula (I) (about 41 mg in each experiment) was mixed with corresponding amounts of each acid to make 1:1 molar ratio. The samples were stirred overnight at room temperature, and XRPD was recorded on the resulting solids after oven drying.

TABLE 18

| | | | | | | | | IPA/Water |
|---|---|---|---|---|---|---|---|---|
| Acids | | Molar | MeOH | EtOAc | EtOH | THF | MeCN | (1:1) |
| (pKa) | MW | Ratio | (1) | (2) | (3) | (4) | (5) | (6) |
| HBr | 80.91 | 1:1 | — | — | — | — | — | — |
| (−9) | | | | | | | | |
| HCl | 36.46 | 1:1 | HCl-1 | HCl-2 | HCl-3 | HCl-4 | HCl-5 | Form F |
| (−6.3) | | | | | | | | |
| H₂SO₄ | 98.08 | 1:1 | SA-1 | Oil | pw* | wax* | Oil | pw* |
| (−3) | | | | | | | chunk* | |
| BSA | 158.2 | 1:1 | Yellow | BSA-1 | BSA-2 | Oil | BSA-3 | Amorphous |
| (−2.8) | | | wax | | | | | |
| MSA | 96.1 | 1:1 | MSA-1 | MSA-2 | MSA-1 | MSA-2 | MSA-2 | Yellow wax |
| (−1.92) | | | | | | | | (MSA-1) |
| ESA | 110.1 | 1:1 | — | — | — | — | — | — |
| (−1.68) | | | | | | | | |
| Maleic | 116.1 | 1:1 | Form L | MAL-1 | MAL-2 | wax | MAL-1 | Yellow wax |
| (1.9) | | | | | | | | |
| Salicylic | 138.1 | 1:1 | Form L | Form B | — | — | — | — |
| (2.97) | | | | | | | | |
| Fumaric | 116.1 | 1:1 | Form L | Form B | — | — | — | — |
| (3.03) | | | | | | | | |
| Citric | 192.1 | 1:1 | Form L | Form B | — | — | — | — |
| (3.13) | | | | | | | | |
| L-Malic | 134.1 | 1:1 | Form L | Form B | — | — | — | — |
| (3.4) | | | | | | | | |

pw: powder
*no further characterization performed
—: not executed
Form B, Form F, and Form L refer to crystalline forms B, F, and L, respectively

Thirteen salt hits of the compound of formula (1) obtained in the salt screen were scaled up using 82 mg of starting material (crystalline form A) in the appropriate solvents. 8 salts were repeated compared to previous data. HCl-2, SA-4, BSA-3, MSA-1 and MSA-2 were not reproduced. Screening conditions with H₂SO₄ were repeated and 4 salt hits were obtained. 5 salts (bold in Table 19) were selected based on crystallinity, repeatability, physical stability (drying, thermal (by DSC and TG), ambient moisture-uptake), and MSA and BSA excluded (see Table 19). An overlay of XRPD diffractograms of the selected salts are shown FIG. 57.

TABLE 19

| | | | | | | IPA:Water |
|---|---|---|---|---|---|---|
| | MeOH | EtOAc | EtOH | THF | MeCN | (1:1) |
| Acids | (1) | (2) | (3) | (4) | (5) | (6) |
| HCl | HCl-1 | Mixture of | HCl-3 | HCl-4′ | HCl-5 | n/a |
| | | crystalline | | | | |
| | | form B and | | | | |
| | | other free | | | | |
| | | forms | | | | |
| H₂SO₄ | SA-1′ | Oil | SA-2 | SA-2 | SA-3 | SA-4 |
| BSA | n/a | BSA-1 | BSA-2 | n/a | BSA-3′ | n/a |
| MSA | n/a | MSA-1′ | n/a | n/a | n/a | MSA-3 |
| Maleic | n/a | ML-1 | ML-2 | n/a | n/a | n/a |

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A crystalline form of a compound of formula (I): 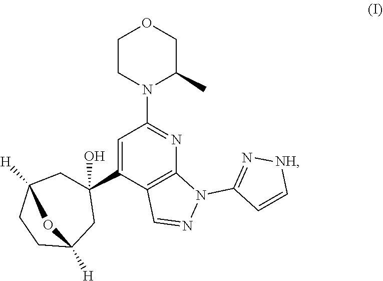
wherein the crystalline form is characterized by a powder x-ray diffraction pattern having peaks at 9.6°2θ±0.2°2θ and 15.8°2θ±0.2°2θ.

2. The crystalline form of claim 1, wherein the crystalline form is further characterized by a powder x-ray diffraction pattern having peaks at 8.9°2θ±0.2°2θ and 20.5°2θ±0.2°2θ.

3. The crystalline form of claim 1 or 2, wherein the crystalline form is further characterized by a powder x-ray diffraction pattern having peaks at 19.8°2θ±0.2°2θ and 20.7°2θ±0.2°2θ.

4. The crystalline form of any one of claims 1 to 3, wherein the crystalline form is further characterized by a powder x-ray diffraction pattern having peaks at 18.2°2θ±0.2 028 and 19.2°2θ±0.2 °2θ.

5. The crystalline form of any one of claims 1 to 4, wherein the crystalline form is further characterized by a powder x-ray diffraction pattern having peaks at 17.8°2θ±0.2°2θ, 22.2°2θ±0.2°2θ, and 23.9°2θ±0.2°2θ.

6. The crystalline form of any one of claims 1 to 5, wherein the crystalline form is further characterized by a differential scanning calorimetry thermogram having an endothermic event onset at 243° C. to 247° C.

7. A crystalline form of a compound of formula (I): 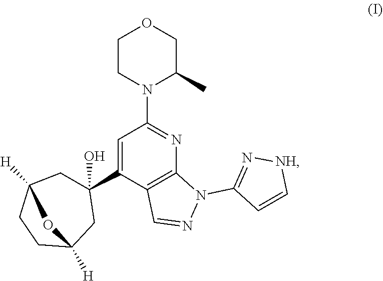
wherein the crystalline form is characterized by a powder x-ray diffraction pattern having peaks at 8.1°2θ±0.2°2θ and 20.3°2θ±0.2°2θ.

8. The crystalline form of claim 7, wherein the crystalline form is further characterized by a powder x-ray diffraction pattern having peaks at 9.1°2θ±0.2°2θ, 15.9°2θ±0.2°2θ, and 23.6°2θ±0.2 °2θ.

9. The crystalline form of claim 7 or 8, wherein the crystalline form is further characterized by a powder x-ray diffraction pattern having peaks at 14.3°2θ±0.2°2θ, 16.8°2θ±0.2°2θ, and 19.3°2θ±0.2 °2θ.

10. The crystalline form of any one of claims 7 to 9, wherein the crystalline form is further characterized by a powder x-ray diffraction pattern having peaks at 16.1°2θ±0.2°2θ and 21.3°2θ±0.2 °2θ.

11. The crystalline form of any one of claims 7 to 10, wherein the crystalline form is further characterized by a powder x-ray diffraction pattern having a peak at 12.3°2θ±0.2°2θ.

12. The crystalline form of any one of claims 7 to 11, wherein the crystalline form is further characterized by a differential scanning calorimetry thermogram having an endothermic event onset at 82° C. to 109° C.

13. The crystalline form of any one of claims 7 to 12, wherein the crystalline form is further characterized by a differential scanning calorimetry thermogram having an endothermic event onset at 245° C. to 248° C.

14. A crystalline form of a compound of formula (I): 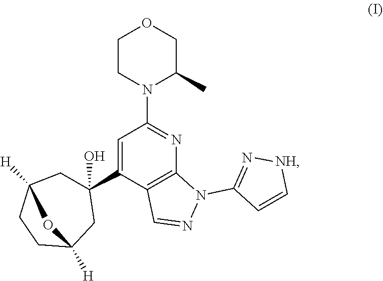
wherein the crystalline form is characterized by a powder x-ray diffraction pattern having peaks at 7.2°2θ±0.2°2θ, 20.4°2θ±0.2°2θ, and 29.4°2θ±0.2°2θ.

15. The crystalline form of claim 14, wherein the crystalline form is further characterized by a powder x-ray diffraction pattern having peaks at 10.2°2θ±0.2°2θ, 14.4°2θ±0.2°2θ, and 17.2°2θ±0.2 °2θ.

16. The crystalline form of claim 14 or 15, wherein the crystalline form is further characterized by a powder x-ray diffraction pattern having peaks at 17.6°2θ±0.2°2θ and 27.0°2θ±0.2°2θ.

17. The crystalline form of any one of claims 14 to 16, wherein the crystalline form is further characterized by a powder x-ray diffraction pattern having peaks at 12.5°2θ±0.2°2θ and 30.9°2θ±0.2 °2θ.

18. The crystalline form of any one of claims 14 to 17, wherein the crystalline form is further characterized by a powder x-ray diffraction pattern having peaks at 15.4°2θ±0.2°2θ and 18.3°2θ±0.2 °2θ.

19. The crystalline form of any one of claims 14 to 18, wherein the crystalline form is further characterized by a powder x-ray diffraction pattern having a peak at 19.7°2θ±0.2°2θ.

20. The crystalline form of any one of claims 14 to 19, wherein the crystalline form is further characterized by a differential scanning calorimetry thermogram having an endothermic event onset at 245° C. to 249° C.

21. The crystalline form of any one of claims 14 to 20, wherein the crystalline form is further characterized by a differential scanning calorimetry thermogram having an endothermic event onset at 111° C. to 154° C.

22. A crystalline form of a hydrogen sulfate salt of a compound of formula (I): 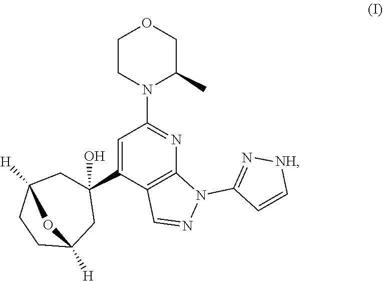
wherein the crystalline form is characterized by a powder x-ray diffraction pattern having peaks at 13.0 °2θ±0.2°2θ, 19.7°2θ±0.2°2θ, and 25.6°2θ±0.2°2θ.

23. The crystalline form of claim 22, wherein the crystalline form is further characterized by a powder x-ray diffraction pattern having peaks at 14.8°2θ±0.2°2θ and 16.5°2θ±0.2°2θ.

24. The crystalline form of claim 22 or 23, wherein the crystalline form is further characterized by a powder x-ray diffraction pattern having peaks at 20.1°2θ±0.2°2θ and 24.5°2θ±0.2°2θ.

25. The crystalline form of any one of claims 22 to 24, wherein the crystalline form is further characterized by a powder x-ray diffraction pattern having peaks at 14.3°2θ±0.2°2θ, 17.9°2θ±0.2°2θ, and 18.2°2θ±0.2°2θ.

26. The crystalline form of any one of claims 22 to 25, wherein the crystalline form is further characterized by a powder x-ray diffraction pattern having peaks at 19.3°2θ±0.2°2θ and 21.4°2θ±0.2 °2θ.

27. The crystalline form of any one of claims 22 to 26, wherein the crystalline form is further characterized by a powder x-ray diffraction pattern having peaks at 24.6°2θ±0.2°2θ and 25.6°2θ±0.2 °2θ.

28. The crystalline form of any one of claims 22 to 27, wherein the crystalline form is further characterized by a differential scanning calorimetry thermogram having an endothermic event onset at 183° C. to 218° C.

29. A crystalline of form of a hydrate of a hydrogen sulfate salt of a compound of formula (I): 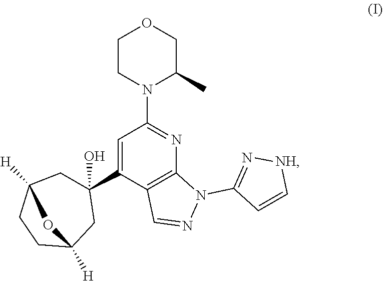
wherein the crystalline form is characterized by a powder x-ray diffraction pattern having peaks at 5.9°2θ±0.2°2θ and 11.8°2θ±0.2°2θ.

30. The crystalline form of claim 29, wherein the crystalline form is further characterized by a powder x-ray diffraction pattern having peaks at 14.9°2θ±0.2°2θ and 20.6°2θ±0.2°2θ.

31. The crystalline form of claim 29 or 30, wherein the crystalline form is further characterized by a powder x-ray diffraction pattern having peaks at 17.2°2θ±0.2°2θ and 21.2°2θ±0.2°2θ.

32. The crystalline form of any one of claims 29 to 31, wherein the crystalline form is further characterized by a powder x-ray diffraction pattern having peaks at 13.3°2θ±0.2°2θ, 14.2°2θ±0.2°2θ, and 22.0°2θ±0.2°2θ.

33. The crystalline form of any one of claims 29 to 32, wherein the crystalline form is further characterized by a powder x-ray diffraction pattern having a peak at 16.7°2θ±0.2°2θ.

34. The crystalline form of any one of claims 29 to 33, wherein the crystalline form is further characterized by a powder x-ray diffraction pattern having peaks at 24.1°2θ±0.2°20 and 29.5°2θ±0.2 °2θ.

35. The crystalline form of any one of claims 29 to 34, wherein the crystalline form is further characterized by a differential scanning calorimetry thermogram having an endothermic event onset at 91° C. to 116° C.

36. A crystalline form of a compound of formula (I): 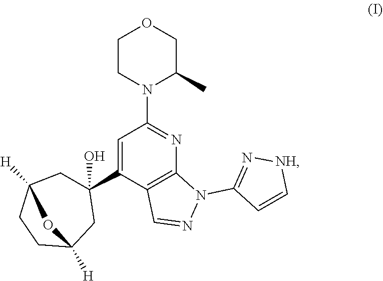
wherein the crystalline form is characterized by a powder x-ray diffraction pattern having peaks at 5.5°2θ±0.2°2θ.

37. The crystalline form of claim 36, wherein the crystalline form is further characterized by a powder x-ray diffraction pattern having a peak at 2.0°2θ±0.2°2θ.

38. The crystalline form of claim 36 or 37, wherein the crystalline form is further characterized by a powder x-ray diffraction pattern having a peak at 8.5°2θ±0.2°2θ.

39. The crystalline form of any one of claims 36 to 38, wherein the crystalline form is further characterized by a powder x-ray diffraction pattern having a peak at 16.2°20 i 0.2°2θ.

40. The crystalline form of any one of claims 36 to 39, wherein the crystalline form is further characterized by a powder x-ray diffraction pattern having peaks at 19.0°2θ±0.2°2θ and 21.1°2θ±0.2 °2θ.

41. The crystalline form of any one of claims 36 to 40, wherein the crystalline form is further characterized by a powder x-ray diffraction pattern having peaks at 12.0°2θ±0.2°2θ and 17.1°2θ±0.2 °2θ.

42. The crystalline form of any one of claims 36 to 41, wherein the crystalline form is further characterized by a powder x-ray diffraction pattern having peaks at 9.5 2±0.2°2θ and 14.7° 2±0.2 °2θ.

43. The crystalline form of any one of claims 36 to 42, wherein the crystalline form is further characterized by a powder x-ray diffraction pattern having a peak at 18.7°20 i 0.2°2θ.

44. The crystalline form of any one of claims 36 to 43, wherein the crystalline form is further characterized by a powder x-ray diffraction pattern having a peak at 18.4°2θ±0.2°2θ.

45. The crystalline form of any one of claims 36 to 44, wherein the crystalline form is further characterized by a differential scanning calorimetry thermogram having an endothermic event onset at 252° C. to 253° C.

46. A crystalline of form of a hemi-sulfate salt of a compound of formula (I): 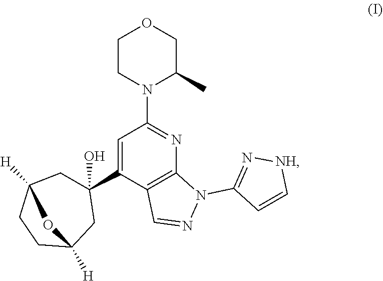
wherein the crystalline form is characterized by a powder x-ray diffraction pattern having peaks at 8.3°2θ±0.2°2θ and 15.6°2θ±0.2°2θ.

47. The crystalline form of claim 46, wherein the crystalline form is further characterized by a powder x-ray diffraction pattern having peaks at 15.1°2θ±0.2°2θ and 23.4°2θ±0.2°2θ.

48. The crystalline form of claim 46 or 47, wherein the crystalline form is further characterized by a powder x-ray diffraction pattern having a peak at 17.4°2θ±0.2°2θ.

49. The crystalline form of any one of claims 46 to 48, wherein the crystalline form is further characterized by a powder x-ray diffraction pattern having a peak at 17.9°2θ i 0.2°2θ.

50. The crystalline form of any one of claims 46 to 49, wherein the crystalline form is further characterized by a differential scanning calorimetry thermogram having an endothermic event onset at 171° C. to 183° C.

51. A hydrogen sulfate salt of the compound of formula (I): 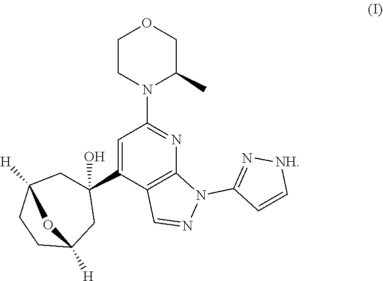
or a hydrate thereof.

52. A hemi-sulfate salt of the compound of formula (I):

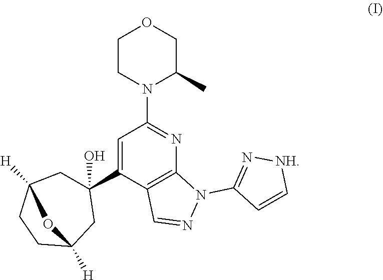

53. A sulfate salt of the compound of formula (I):

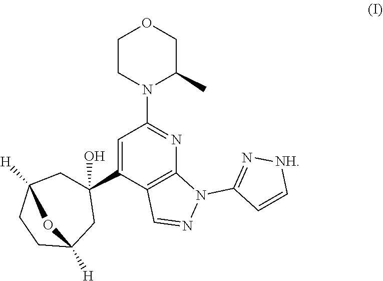

54. A pharmaceutical composition comprising the crystalline form of any one of claims 1 to 50 or the salt of any one of claims 51 to 53.

55. A method of inhibiting ATR kinase in a cell expressing ATR kinase, the method comprising contacting the cell with an effective amount of the crystalline form of any one of claims 1 to 50, the pharmaceutical composition of claim 54, or the salt of any one of claims 51 to 53.

56. The method of claim 55, wherein the cell is in a subject.

57. A method of treating a subject in need thereof comprising administering to the subject an effective amount of the crystalline form of any one of claims 1 to 50, the pharmaceutical composition of claim 54, or the salt of any one of claims 51 to 53.

58. The method of claim 57, wherein the subject is suffering from, and is in need of a treatment for, a disease or condition having the symptom of cell hyperproliferation.

59. The method of claim 58, wherein the disease or condition is a cancer.

60. The method of claim 59, wherein the cancer is a carcinoma, sarcoma, adenocarcinoma, leukemia, or melanoma.

61. The method of claim 59, wherein the cancer is a carcinoma selected from the group consisting of medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, Schneiderian carcinoma, scirrhous carcinoma, carcinoma scroti, signet ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

62. The method of claim 59, wherein the cancer is a sarcoma selected from the group consisting of chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telang iectaltic sarcoma.

63. The method of claim 59, wherein the cancer is a leukemia selected from the group consisting of nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

64. The method of claim 59, wherein the cancer is a melanoma selected from the group consisting of acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungual melanoma, and superficial spreading melanoma.

65. The method of claim 59, wherein the cancer is prostate cancer, thyroid cancer, endocrine system cancer, brain cancer, breast cancer, cervix cancer, colon cancer, head & neck cancer, liver cancer, kidney cancer, lung cancer, non-small cell lung cancer, melanoma, mesothelioma, ovarian cancer, sarcoma, stomach cancer, uterus cancer, medulloblastoma, ampullary cancer, colorectal cancer, or pancreatic cancer.

66. The method of claim 59, wherein the cancer is Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulinoma, malignant carcinoid, urinary bladder cancer, premalignant skin lesions, testicular cancer, lymphoma, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

67. The method of claim 59, wherein the subject is suffering from, and is in need of a treatment for, a pre-malignant condition.
